(19) United States
(12) Patent Application Publication
(10) Pub. No.: US 2023/0173030 A1
(43) Pub. Date: Jun. 8, 2023

(54) VASOACTIVE INTESTINAL PEPTIDE FUSION PROTEINS FOR THE TREATMENT OF COVID-19

(71) Applicant: PhaseBio Pharmaceuticals, Inc., Malvern, PA (US)

(72) Inventors: John LEE, Malvern, PA (US); Paul SUMITA, Malvern, PA (US)

(21) Appl. No.: 17/997,512

(22) PCT Filed: May 5, 2021
(86) PCT No.: PCT/US21/30901

§ 371 Date: Oct. 28, 2022

Related U.S. Application Data
(60) Provisional application No. 63/020,468, filed on May 5, 2020.
(60) Provisional application No. 63/032,244, filed on May 29, 2020.

(51) Int. Cl. A61K 38/22; A61K 38/39; A61P 31/14

(52) U.S. Cl. CPC A61K 38/2278; A61K 38/39; A61P 31/14

(57) ABSTRACT

The present disclosure provides methods of treating inflammatory lung conditions such as COVID-19 with a fusion protein comprising a vasoactive intestinal peptide (VIP) and an elastin-like peptide.

### CROSS-REFERENCE TO RELATED APPLICATIONS

This application claims priority to U.S. Provisional Application No. 63/020,468 filed May 5, 2020 and U.S. Provisional Application No. 63/032,244 filed May 29, 2020, the contents of each of which are incorporated by reference in their entireties for all purposes.

### DESCRIPTION OF THE TEXT FILE SUBMITTED ELECTRONICALLY

The contents of the text file submitted electronically herewith are incorporated herein by reference in their entirety: a computer readable format copy of the sequence listing (filename: PHAS_042_0WO_SeqList_ST25.txt, date recorded: May 5, 2021, file size 23 kilobytes).

### BACKGROUND

The global pandemic COVID-19 is caused by the novel coronavirus SARS-CoV-2. The clinical profile of COVID-19 patients ranges from asymptomatic or mild respiratory symptoms to severe progressive pneumonia, leading to hypoxic respiratory failure and death in up to 50% of patients admitted to the intensive care unit for mechanical ventilation (Wu et al., 2020). Acute respiratory distress syndrome (ARDS) is a severe complication of hypoxemic respiratory failure caused by COVID-19 and other critical respiratory conditions. ARDS is characterized by tissue injury at the alveolar-capillary membrane, leading to diffuse inflammation, pulmonary edema, and intractable hypoxemic respiratory failure. There are 3 phases of ARDS: 1) an initial inflammatory phase, 2) a fibroproliferative phase, and 3) a final fibrotic phase. Currently there is no pharmacological intervention for treatment of patients with ARDS. Supportive care and mechanical ventilation in intensive care units is the current standard of care for ARDS patients, including those with COVID-19. ARDS mortality overall is approximately 40% (Zhang et al., 2019). In a recent COVID-19 case series describing 201 patients with COVID-19 in Wuhan China, 41.8% of patients developed ARDS, and of those, 52.4% died (Wu et al., 2020). In a case series of 5700 COVID-19 patients admitted to twelve hospitals in the New York city area, mortality for those requiring mechanical ventilation was 88% (Richardson et al., 2020). There is a critical need for pharmacological intervention for patients at high risk of COVID-19-related ARDS.

### SUMMARY OF THE INVENTION

The present disclosure provides a Vasoactive Intestinal Peptide (VIP) therapeutic for prevention or treatment of ARDS, including in patients with COVID-19. This therapeutic, PB1046 is an investigational compound comprising the neuropeptide, Vasoactive Intestinal Peptide (VIP) genetically fused to an elastin-like polypeptide biopolymer (ELP). PB1046 is being developed as an adjunctive therapy for the treatment of hospitalized COVID-19 patients at high risk for rapid deterioration, mechanical ventilation, ARDS, and death. PB1046 is also being developed as adjunctive therapy for Pulmonary Arterial Hypertension (PAH).

In some embodiments, the present disclosure provides methods of treating inflammatory lung disease comprising administering a pharmaceutical composition comprising Vasoactive Intestinal Peptide (VIP) and an elastin-like peptide (ELP) to a patient in need thereof. In some embodiments, the present disclosure provides methods of treating inflammatory heart disease comprising administering a pharmaceutical composition comprising Vasoactive Intestinal Peptide (VIP) and an elastin-like peptide (ELP) to a patient in need thereof. In some embodiments, the pharmaceutical composition comprises the polypeptide of SEQ ID NO: 3.

In some embodiments, the patient is infected by, or presumed to be infected by, SARS-CoV-2. In some embodiments, the patient has developed COVID-19 or symptoms thereof. In some embodiments, the patient has developed severe COVID-19 or symptoms thereof. In some embodiments, the patient has one or more symptoms of ARDS, but is not infected by SARS-CoV-2. In some embodiments, the patient is at risk of developing ARDS, but is not infected by SARS-CoV-2.

In some aspects, the present disclosure provides methods of treating a patient exhibiting one or more symptoms of SARS-CoV-2 infection, comprising administering an effective amount of a pharmaceutical composition comprising a VIP peptide and an elastin-like peptide (ELP). In some embodiments, the pharmaceutical composition is administered prior to the development of Acute Respiratory Distress Syndrome (ARDS) in the patient. In some embodiments, the pharmaceutical composition is administered when the patient is exhibiting one or more symptoms of ARDS. In some embodiments, administration of the pharmaceutical composition prevents the onset or progression of ARDS in the patient.

In some embodiments, the patient is at high risk of developing severe COVID-19, ARDS, or symptoms thereof. In some embodiments, the patient presents with a comorbidity. In some embodiments, the comorbidity increases the risk of the patient developing severe COVID-19, ARDS, or symptoms thereof. In some embodiments, the comorbidity is selected from the group consisting of: obesity, hypertension, diabetes, an autoimmune disorder (e.g. rheumatoid arthritis), heart disease, heart failure, atherosclerosis, cancer (e.g. lung cancer), a history of smoking or exposure to other lung-damaging agents), liver disease, alcoholism, other pulmonary infection, and chronic kidney disease. In some embodiments, the patient presents with elevated markers of cardiac injury or dysfunction.

In some embodiments, one or more factors increasing patient risk of developing severe COVID-19 is race and/or socioeconomic status.

In some embodiments, the patient presents with one or more of the following symptoms:

a) low oxygen saturation levels;

b) increased respiration rate;

c) requires oxygen therapy;

d) requires a ventilator to breathe; and

e) fever.

In some aspects, the present disclosure provides methods of treating a patient exhibiting one or more symptoms of SARS-CoV-2 infection, comprising administering and effective amount of a pharmaceutical composition comprising a VIP peptide and an ELP. In some embodiments, the pharmaceutical composition is administered prior to the development of Acute Respiratory Distress Syndrome (ARDS) in the patient. In some embodiments, the pharmaceutical composition is administered when the patient is exhibiting one or more symptoms of ARDS. In some embodiments, administration of the pharmaceutical composition prevents the onset or progression of ARDS in the patient.

In some embodiments, the patient is administered a low dose of the pharmaceutical composition comprising the polypeptide of SEQ ID NO: 3. In some embodiments, the patient is administered a dose of about 10 mg of the pharmaceutical composition comprising the polypeptide of SEQ ID NO: 3. In some embodiments, the patient is administered a low dose of the pharmaceutical composition comprising the polypeptide of SEQ ID NO: 3 for four weeks or until hospital discharge.

In some embodiments, the patient is administered a moderate dose of the pharmaceutical composition comprising the polypeptide of SEQ ID NO: 3. In some embodiments, the patient is administered a dose of about 40 mg of the pharmaceutical composition comprising the polypeptide of SEQ ID NO: 3. In some embodiments, the patient is administered a moderate dose of the pharmaceutical composition comprising the polypeptide of SEQ ID NO: 3 for four weeks or until hospital discharge.

In some embodiments, the patient is administered a high dose of the pharmaceutical composition comprising the polypeptide of SEQ ID NO: 3. In some embodiments, the patient is administered a dose of about 100 mg of the pharmaceutical composition comprising the polypeptide of SEQ ID NO: 3. In some embodiments, the patient is administered a high dose of the pharmaceutical composition comprising the polypeptide of SEQ ID NO: 3 for four weeks or until hospital discharge.

In some embodiments, the pharmaceutical composition comprises the polypeptide of SEQ ID NO: 3 is administered subcutaneously.

### DETAILED DESCRIPTION

VIP is a peptide hormone produced in many tissues throughout the body. Native VIP exerts its function in the body by binding to two distinct receptors: vasoactive intestinal peptide receptor 1, or VPAC1, and vasoactive intestinal peptide receptor 2, or VPAC2. As is the case for many other peptide hormones, the body uses VIP for distinct purposes in different locations. VPAC1 is found predominantly in the gastrointestinal tract, while VPAC2 is found predominantly in the myocardial wall and pulmonary arteries.

Vasoactive intestinal peptide (VIP) is a neuropeptide that is highly conserved across multiple vertebrate species and exhibits physiologic effects that may be beneficial in COVID-19 patients. Human VIP (SEQ ID NO: 5) is a 28 amino acid peptide that interacts with VIP-specific receptors (VPAC1 and VPAC2) that are widely distributed throughout the pulmonary and peripheral vasculature, the ventricular myocardium, and a broad range of inflammatory cell types. The physiologic effects of VIP vary according to the location of its receptors. In the arterial vasculature VIP has vasodilatory properties, whereas in the right and left ventricles, VIP potentiates inotropy and lusitropy, leading to increases in cardiac output in animal models with no net increase in oxygen consumption. For patients with inflammatory pulmonary disease, including COVID-19, VIP has immunomodulatory effects that may provide a novel approach to treatment when added to standard of care therapy.

VIP has multiple anti-inflammatory and immune-protective properties, including upregulation of anti-inflammatory cytokine IL-10 and induction of protective T cells (Szema & Hamidi, 2014). VIP is an important regulator of many cytokines, including TNFα, IL-1β, and IL-6 (Delgado et al., 1999) that are believed to contribute to cytokine-mediated acute lung injury and ARDS. In rodent animal models, VIP demonstrated suppression of pulmonary inflammation mediated by alveolar macrophages and exerted antioxidant effects on free radicals present during the inflammatory response (Berisha et al., 1990; Sakakibara et al., 1994). VIP has also been shown to inhibit the pro-inflammatory cytokine IL-17A, which has been implicated in the inflammatory response to acute lung injury (Ran et al., 2015). In animal models of acute lung injury (ALI), which exhibit inflammation in the pulmonary endothelium, epithelium, and capillaries, VIP inhibited cytokine release and prevented damage to lung tissue by downregulating the potent proinflammatory cytokines IL-17A and TNTα (Ran et al., 2015; Sun et al., 2018). Other models of ALI in mice have shown that VIP inhibited the pro-inflammatory mediators NF-κB and NLRP3 (Zhou 2020). Recently VIP was found to inhibit fibroblast formation, which occurs during the fibroproliferative phase of ARDS, by downregulation of IL-17 receptor C (Zhang et al., 2019).

In humans, VIP has been tested in multiple clinical trials, demonstrating improvements in lung function, exercise capacity, and quality of life in patients with pulmonary arterial hypertension (PAH) and COPD (Petkov et al., 2003; Burian et at, 2006), using an aerosolized formulation in Phase 1 and 2 studies. In an open-label pilot study of an intravenous formulation of human VIP (aviptadil), treatment of 8 patients with sepsis-related ARDS was associated with successful extubation of 7 of 8 mechanically ventilated patients (NCT00004494, unpublished data).

Native VIP is rapidly degraded, and, when injected into the body, is eliminated within minutes, limiting its therapeutic effect. High levels of native VIP also result in severe gastrointestinal problems due to VPAC1 activation. However, the fusion of a VIP with an ELP technology extends the half-life of the VIP molecule present in PB1046 to approximately 60 hours.

PB1046 is active predominantly on VPAC2 rather than VPAC1 in order to preferentially affect the lung and cardiac tissue and reduce the potential for gastrointestinal side effects associated with VPAC1 activation.

PB1046, a subcutaneously injected sustained-release analogue of the native human peptide VIP, has demonstrated dose-dependent phartnacodynamic effects of VIP and a PK profile supportive of weekly subcutaneous dosing in hospitalized COVID-19 patients. In Phase 1 single- and multiple-dose studies, PB1046 was found to be safe and generally well tolerated with no reported events of symptomatic hypotension. PB1046 is currently being investigated in a human Phase 2b clinical trial in patients with PAH as adjunctive therapy added to PAH standard of care. PB1046 has received orphan designation for both PAH and DMD-associated cardiomyopathy.

As a stable, long-acting VIP analogue, PB1046 may provide an attractive potential therapy for COVID-19 patients who are at high risk for rapid clinical deterioration due to pulmonary inflammation and ARDS (Wu et al., 2020; Mehta et al., 2020). The pulmonary immunomodulatory effects of PB1046 suppresses cytokine-mediated inflammatory responses, potentially leading to significant clinical improvement compared to current standard of care supportive measures.

### Vasoactive Intestinal Peptides

In some aspects the disclosure provides therapeutic compositions that include a modified VIP peptide. In some embodiments, the therapeutic compositions include a VPAC-2 selective VIP fused to an elastin-like peptide. In some embodiments, the therapeutic composition is a polypeptide comprising the amino acid sequence of SEQ ID NO: 2. In some embodiments, the therapeutic composition is a polypeptide comprising the amino acid sequence of SEQ ID NO: 8. In some embodiments, the therapeutic composition is a polypeptide comprising the amino acid sequences of both SEQ ID NO: 2 and SEQ ID NO: 8. In some embodiments, the therapeutic composition is a polypeptide comprising the amino acid sequence of SEQ ID NO: 3. In some embodiments, the therapeutic composition is a polypeptide consisting of the amino acid sequence of SEQ ID NO: 3.

In some embodiments, the modified VIP peptide contains one or more amino acid substitutions compared to the amino acid sequence of mature VIP (e.g. SEQ ID NO: 5). In some embodiments, one to 20 amino acids are substituted compared to the amino acid sequence of mature VIP (SEQ ID NO: 5). In some embodiments, the modified VIP peptide contains about 1, about 2, about 3, about 4, about 5, about 6, about 7, about 8, about 9, or about 10 amino acid substitutions compared to the amino acid sequence of mature VIP (SEQ ID NO: 5).

In some embodiments, the modified VIP peptide contains one or more amino acid deletions compared to the amino acid sequence of mature VIP (SEQ ID NO: 3). In some embodiments, one to 20 amino acids are deleted compared to the amino acid sequence of mature VIP (SEQ ID NO: 517). In some embodiments, the modified VIP peptide has about 1, about 2, about 3, about 4, about 5, about 6, about 7, about 8, about 9, or about 10 amino acid deletions compared to the amino acid sequence of mature VIP (SEQ ID NO: 5). In some embodiments, one to ten amino acids are deleted at either terminus compared to the amino acid sequence of mature VIP (SEQ ID NO: 5). In some embodiments, one to ten amino acids are deleted from both termini compared to the amino acid sequence of mature VIP (SEQ ID NO: 5). In some embodiments, the amino acid sequence of the modified VIP peptide is at least about 70% identical to the amino acid sequence of mature VIP (SEQ ID NO: 5). In some embodiments, the amino acid sequence of the modified VIP peptide is about 70%, about 80%, about 85%, about 90%, about 95%, about 96%, or about 97% identical to the amino acid sequence of mature VIP (SEQ ID NO: 5). Percentage identity can be calculated using the alignment program ClustalW2, available at http://www.ebi.ac.uk/Tools/psa/emboss_needle/. The following default parameters may be used for Pairwise alignment: Protein Weight Matrix=BLOSUM62; Gap Open=10, Gap Extension=0.1.

In various aspects, the present disclosure provides a modified VIP peptide having relative receptor preference for VPAC2 or VPAC1, as compared to mature VIP (i.e., SEQ ID NO: 5). For example, the modified VIP peptide may have a relative binding preference for VPAC2 over VPAC1 of at least about 2:1, about 5:1, about 10:1, about 25:1, about 50:1, about 100:1, about 500:1 or more. In other embodiments, the modified VIP peptide may have a relative binding preference for VPAC1 over VPAC2 of at least about 2:1, about 5:1, about 10:1, about 25:1, about 50:1, about 100:1, about 500:1, or more. For example, in certain embodiments, the modified VIP peptide activates the VPAC2 receptor with an EC50 within a factor of about 2-4 of mature human VIP (SEQ ID NO: 5). However, in some embodiments, this same modified VIP peptide is 50- or 100-fold or more less potent than mature, unmodified, human VIP peptide (SEQ ID NO: 5) in activating the VPAC1 receptor.

In some embodiments, the modified VIP peptide contains additional amino acid residues compared to mature VIP (SEQ ID NO: 5). In some embodiments, the modified VIP peptide contains one or more amino acids added at the N- and/or C-terminus compared to mature VIP (SEQ ID NO: 5). Such modified VIP peptides may contain modified N-terminal regions, such as an addition of from 1 to about 500 amino acids to the N-terminal histidine of VIP, which may include heterologous mammalian (e.g. non-human) amino acid sequences. The additional sequence added to the N-terminus of VIP may be of any sequence, including biologically active and biologically inert sequences of from 1 to about 100, 1 to about 50, 1 to about 20, 1 to about 10, and 1 to about 5 amino acids. For example, the modified VIP may contain a single methionine at the N-terminal end of the natural N-terminal histidine of mature VIP. While methionine can sometimes be removed by methionine aminopeptidase (MA) in bacterial expression systems, histidine (H) is one of the least favored residues at position 2 for MA. In some embodiments, the modified VIP peptide is SEQ ID NO: 2. Such modified VIP peptides containing an N-terminal methionine can be prepared in E. coli or other bacterial or yeast expression systems, since the methionine will not be removed by E. coli when the adjacent amino acid is histidine. Alternatively, the N-terminal amino acid may be any of the naturally-occurring amino acids, namely alanine, arginine, asparagine, aspartic acid, cysteine, glutamic acid, glutamine, glycine, histidine, isoleucine, leucine, lysine, methionine, phenylalanine, serine, threonine, tryptophan, tyrosine, valine, and proline. In other embodiments, the VIP peptide is activatable by a peptidase or protease, such as an endogenous peptidase or protease. Such activatable sequences are described, for example, in International Application No. PCT/US2009/068656. As used herein, the terms “peptidase” and “protease” are interchangeable. For example, the VIP peptide may be designed to be activatable by a dipeptidyl peptidase. Exemplary dipeptidyl peptidases include dipeptidyl peptidase-1 (DPP-I), dipeptidyl peptidase-3 (DPP-III), dipeptidyl peptidase-4 (DPP-IV), dipeptidyl peptidase-6 (DPP-VI), dipeptidyl peptidase-7 (DPP-VII), dipeptidyl peptidase-8 (DPP-VIII), dipeptidyl peptidase-9 (DPP-IX), dipeptidyl peptidase-10 (DPP-X). Substrate sequences for such dipeptidases are known.

In some embodiments, the N-terminus of an activatable VIP peptide may have the structure Z-N, where Z is a substrate for a dipeptidase (e.g., Z is removed by dipeptidase exposure), and N is the N-terminus of VIP. The activatable VIP peptide may have an N-terminal sequence with the formula M-X-N where M is methionine, X is Pro, Ala, or Ser, and N is the N-terminal of VIP or VIP analog. In this manner, M and X will be sensitive to, and removed by a host cell (e.g., E. coli.), and/or a dipeptidase (e.g., DPP-IV), subsequently. Alternatively, the N-terminal sequence of the activatable VIP may be X1-X2-N, where X1 is Lily, Ala, Ser, Cys, Thr, Val, or Pro; X2 is Pro, Ala, or Ser; and N is the N-terminal of VIP. X1-X2 is a substrate for dipeptidase (e.g., DPP-IV), and dipeptidase digestion will expose N, the desired N-terminus of the VIP or the VIP analog. In such embodiments, the VIP peptide may be produced by expression of a construct encoding M-X1-X2-N (where M is methionine) in a host cell (e.g., E. coli), since Gly, Ala, Ser, Cys, Thr, Val, or Pro at the second position will signal the removal of the Met, thereby leaving X1-X2 on the N-terminus, which can be activated by a dipeptidase (e.g., DPP-IV) in vivo. In some embodiments, the peptidase may be present in the body and act on the activatable VIP peptide after injection. In some embodiments, the activatable VIP peptide contains the amino acid sequence MAA added at the N-terminus compared to mature VIP (e.g. SEQ ID NO: 5). In some embodiments, the activatable VIP peptide is SEQ ID NO: 4.

In other embodiments, the N-terminus of the modified activatable VIP peptide has the structure M-Z-N, where M is methionine, Z is a substrate for a dipeptidase (e.g., Z is removed by dipeptidase exposure), and N is a non-His N-terminal of an activatable VIP. For example, the modified activatable VIP peptide may have an N-terminal sequence with the formula M-X-N where M is methionine; X is Pro, Ala, or Ser; and N is a non-His N-terminal of the activatable VIP. In this manner, NT and X will be sensitive to, and removed by a host cell (e.g., E. coli), and/or a dipeptidase (e.g., DPP-IV), subsequently. Alternatively, the N-terminal sequence of the activatable VIP peptide may be X1-X2-N, where X1 is Gly, Ala, Ser, Cys, Thr, Val, or Pro; X2 is Pro, Ala, or Ser; and N is a non-His N-terminal of the activatable VIP. X1-X2 is a substrate for dipeptidase (e.g., DPP-IV), and dipeptidase digestion will expose N, the desired non-His N-terminus of the VIP.

Still other embodiments, the N-terminus of an activatable VIP peptide has the structure M-Z-S-N, where M is methionine; Z is a substrate for a dipeptidase (e.g., Z is removed by dipeptidase exposure); N is the N-terminus of mature VIP (His); and S is one or more amino acids which will be exposed after dipeptidase digestion, and which provide an activatable VIP as previously described. For example, the activatable VIP peptide may have an N-terminal sequence with the formula M-X-S-N where M is methionine, X is Pro, Ala, or Ser; N is the N-terminal of mature VIP (e.g. SEQ ID NO: 5); and S is one or more amino acids which will be exposed after dipeptidase digestion, and will provide receptor preference. Alternatively, the N-terminal sequence of the activatable VIP peptide may be X1-X2-S-N, where X1 is Gly, Ala, Ser, Cys, Thr, Val, or Pro; X2 is Pro, Ala, or Ser; N is a non-His N-terminal of VIP; and S is one or more amino acids which will be exposed after dipeptidase digestion. X1-X2 is a substrate for dipeptidase (e.g., DPP-IV), and dipeptidase digestion will expose S.

In still other embodiments, the VIP peptide is modified by fusion with a mammalian heterologous protein, such as a mammalian protein effective for extending half-life of therapeutic molecules. Such sequences may be mammalian sequences, such as albumin, transferrin, or antibody Fc sequences. Such sequences are described in U.S. Pat. No. 7,238,667 (particularly with respect to albumin fusions), U.S. Pat. No. 7,176,278 (particularly with respect to transferrin fusions), and U.S. Pat. No. 5,766,883. In some embodiments, the VIP peptide is modified by fusion with a mammalian heterologous protein at the N-terminus. In some embodiments, the VIP is modified by fusion with a mammalian heterologous protein at the C-terminus. In some embodiments, the VIP is modified by fusion with a mammalian heterologous protein at both the N- and C-termini.

In some embodiments, N-terminal chemical modifications to the VIP peptide N-terminus provides receptor preference. Chemical modification of proteins and methods thereof are well known in the art. Non-limiting exemplary chemical modifications are PEGylation, methylglyoxalation, reductive alkylation, performic acid oxidation, succinylation, aminoethylation, and lipidation (Clifton, New Protein Techniques, N.J.: Humana Press, 1985. ISBX. 0-89603-126-8. Volume. 3 of. Methods in Molecular Biology). Chemical groups, such as PEGylation, may be attached by modifications of cysteine, methionine, histidine, lysine, arginine, tryptophan, tyrosine, carboxyl groups have been described previously (see Lindblad, Techniques in Protein Modification, CRC Press, 1995).

In still other embodiments, the VIP peptide is modified by fusion with a protein including a repeating amino acid sequence, such as a sequence comprising prolines, alanines, and serines (e.g. PASylation (Schlapschy, M. et al. (2013)), or XTEN sequences (Schellenberger, V. et al. (2009)).

### Elastin-Like Peptides

In some aspects the disclosure provides therapeutic compositions that include a Vasoactive Intestinal Peptide and one or more elastin-like peptides (ELP). In some embodiments, a VIP peptide and one or more ELPs are fused together. In some embodiments, a VIP peptide and one or more ELPs are produced as a recombinant fusion polypeptide. In some embodiments, the therapeutic composition includes a Vasoactive Intestinal Peptide and one or more ELPs as separate molecules. In yet other embodiments, the compositions include a VIP-ELP fusion protein and ELPs as separate molecules. In some embodiments, the compositions include SEQ ID NO: 3. In some embodiments, the compositions include SEQ ID NO: 7. In some embodiments, the compositions include SEQ ID NO: 4.

The ELP sequence includes structural peptide units or sequences that are related to, or mimics of, the elastin protein. The ELP sequence is constructed from structural units of from three to about twenty amino acids, or in some embodiments, from four to ten amino acids, such as four, five or six amino acids. The length of the individual structural units may vary or may be uniform. For example, structural units include units defined by SEQ ID NO: 1, which may be employed as repeating structural units, including tandem-repeating units.

In some embodiments, the amino acid sequence of the ELP unit is from about 1 to about 500 structural units, or in certain embodiments about 9 to about 200 structural units, or in certain embodiments about 10 to 200 structural units, or in certain embodiments about 50 to about 200 structural units, or in certain embodiments from about 80 to about 200 structural units, or from about 80 to about 150 structural units, such as units defined by SEQ ID NO: 1. Thus, the structural units collectively may have a length of from about 50 to about 2000 amino acid residues, or from about 100 to about 800 amino acid residues, or from about 200 to about 700 amino acid residues, or from about 400 to about 600 amino acid residues. In exemplary embodiments, the amino acid sequence of the ELP structural unit includes about 3 structural units, about 7 structural units, about 9 structural units, about 10 structural units, about 15 structural units, about 20 structural units, about 40 structural units, about 80 structural units, about 90 structural units, about 100 structural units, about 120 structural units, about 140 structural units, about 144 structural units, about 160 structural units, about 180 structural units, about 200 structural units, or about 500 structural units. In exemplary embodiments, the structural units collectively have a length of about 45 amino acid residues, of about 90 amino acid residues, of about 100 amino acid residues, of about 200 amino acid residues, of about 300 amino acid residues, of about 400 amino acid residues, of about 500 amino acid residues, of about 600 amino acid residues, of about 700 amino acid residues, of about 720 amino acid residues, of about 800 amino acid residues, or of about 1000 amino acid residues.

The ELP amino acid sequence may exhibit a visible and reversible inverse phase transition with the selected formulation. That is, the ELP amino acid sequence may be structurally disordered and highly soluble in the formulation below a transition temperature (Tt), but exhibit a sharp (2-3° C. range) disorder-to-order phase transition when the temperature of the formulation is raised above the Tt. In addition to temperature, length of the amino acid polymer, amino acid composition, ionic strength, pH, pressure, temperature, selected solvents, presence of organic solutes, and protein concentration may also affect the transition properties, and these may be tailored in the formulation for the desired absorption profile. The absorption profile can be easily tested by determining plasma concentration or activity of the active agent over time.

In certain embodiments, the ELP component(s) may be formed of the pentapeptide Val-Pro-Gly-X-Gly (SEQ ID NO: 1), or VPGXG, where X is any natural or non-natural amino acid residue, and where X optionally varies among polymeric or oligomeric repeats.

In certain embodiments, the ELP contains repeat units, including tandem repeating units, of Val-Pro-Gly-X-Gly (SEQ ID NO: 1), where X is as defined above, and where the percentage of Val-Pro-Gly-X-Gly units taken with respect to the entire ELP component (which may comprise structural units other than VPGXG) is greater than about 50%, or greater than about 75%, or greater than about 85%, or greater than about 95% of the ELP. The ELP may contain motifs of 5 to 15 structural units (e.g. about 10 structural units) of SEQ ID NO: 1, with the guest residue X varying among at least 2 or at least 3 of the units in the motif. The guest residues may be independently selected, such as from non-polar or hydrophobic residues, such as the amino acids V, I, L, A, G, and W (and may be selected so as to retain a desired inverse phase transition property). In certain embodiments, the guest residues are selected from V, G, and A. In some embodiments, the ELP includes the ELP 1 series (VPGXG: V5A2G3). In some embodiments, the ELP includes the amino acid sequence of SEQ ID NO: 8.

In certain embodiments, the ELP is the ELP-1 series which includes [VPGXG]ₘ, where m is any number from 1 to 200, each X is selected from V, G, and A, and wherein the ratio of V:G:A may be about 5:3:2. In certain embodiments, ELP includes [VPGXG]₉₀, where each X is selected from V, G, and A, and wherein the ratio of V:G:A may be about 5:3:2. In certain embodiments, the ELP includes [VPGXG]₁₂₀, where each X is selected from V, G, and A, and wherein the ratio of V:G:A may be about 5:3:2.

In some embodiments, the ELP may form a β-turn structure. Exemplary peptide sequences suitable for creating a β-turn structure are described in International Patent Application PCT/US96/05186. For example, the fourth residue (X) in the sequence VPGXG, can be altered without eliminating the formation of a β-turn.

The structure of exemplary ELPs may be described using the notation ELPk [XᵢYⱼ-n], where k designates a particular ELP repeat unit, the bracketed capital letters are single letter amino acid codes and their corresponding subscripts designate the relative ratio of each guest residue X in the structural units (where applicable), and n describes the total length of the ELP in number of the structural repeats. For example, ELP1 [V₅A₂G₅-10] designates an ELP component containing 10 repeating units of the pentapeptide VPGXG, where X is valine, alanine, and glycine at a relative ratio of about 5:2:3.

With respect to the ELP, the Tt is a function of the hydrophobicity of the guest residue. Thus, by varying the identity of the guest residue(s) and their mole fraction(s), ELPs can be synthesized that exhibit an inverse transition over a broad range. Thus, the Tt at a given ELP length may be decreased by incorporating a larger fraction of hydrophobic guest residues in the ELP sequence. Examples of suitable hydrophobic guest residues include valine, leucine, isoleucine, phenylalanine, tryptophan and methionine. Tyrosine, which is moderately hydrophobic, may also be used. Conversely, the Tt may be increased by incorporating residues, such as those selected from: glutamic acid, cysteine, lysine, aspartate, alanine, asparagine, serine, threonine, glycine, arginine, and glutamine.

For polypeptides having a molecular weight>100,000 Da, the hydrophobicity scale disclosed in PCT/US96/05186 provides one means for predicting the approximate Tt of a specific ELP sequence. For polypeptides having a molecular weight<100,000 Da, the Tt may be predicted or determined by the following quadratic function: Tt=M0+M1X+M2X2 where X is the MW of the fusion protein, and M0=116.21; M1=−1.7499; M2=0.010349.

The ELP in some embodiments is selected or designed to provide a Tt ranging from about 10 to about 37° C. at formulation conditions, such as from about 20 to about 37° C., or from about 25 to about 37° C. In some embodiments, the transition temperature at physiological conditions (e.g., 0.9% saline) is from about 34 to 36° C., to take into account a slightly lower peripheral temperature.

Elastin-like-peptide (ELP) protein polymers and recombinant fusion proteins can be prepared as described in U.S. Patent Publication No. 2010/0022455. In some embodiments, the ELPs are constructed through recursive ligation to rapidly clone highly repetitive polypeptides of any sequence and specified length over a large range of molecular weights. In a single cycle, two halves of a parent plasmid, each containing a copy of an oligomer, are ligated together, thereby dimerizing the oligomer and reconstituting a functional plasmid. This process is carried out recursively to assemble an oligomeric gene with the desired number of repeats. For example, one ELP structural subunit (e.g. a pentapeptide or a 9mer of pentapeptides) is inserted into a vector. The vector is digested, and another ELP structural unit (e.g. a pentapeptide or a 9mer of pentapeptides) is inserted. Each subsequent round of digestion and ligation doubles the number of ELP structural units contained in the resulting vector until the ELP polymer is the desired length.

In other embodiments, the ELP includes a random coil or non-globular extended structure. For example, the ELP includes an amino acid sequence disclosed in U.S. Patent Publication No. 2008/0286808, WIPO Patent Publication No. 2008/155134, and U.S. Patent Publication No. 2011/0123487.

For example, in some embodiments the ELP amino acid sequence includes an unstructured recombinant polymer of at least 40 amino acids. For example, the unstructured polymer may be defined where the sum of glycine (G), aspartate (D), alanine (A), serine (S), threonine (T), glutamate (E) and proline (P) residues contained in the unstructured polymer, constitutes more than about 80% of the total amino acids. In some embodiments, at least 50% of the amino acids are devoid of secondary structure as determined by the Chou-Fasman algorithm. The unstructured polymer includes more than about 100, 150, 200 or more contiguous amino acids. In some embodiments, the amino acid sequence forms a random coil domain. In particular, a polypeptide or amino acid polymer having or forming “random coil conformation” substantially lacks a defined secondary and tertiary structure.

In various embodiments, the intended subject is human, and the body temperature is about 37° C., and thus the therapeutic agent is designed to provide a sustained release at or near this temperature (e.g. between about 28° C. to about 37° C.). A slow release into the circulation with reversal of hydrogen bonding and/or hydrophobic interactions is driven by a drop in concentration as the product diffuses at the injection site, even though body temperature remains constant. In other embodiments, the subject is a non-human mammal, and the therapeutic agent is designed to exhibit a sustained release at the body temperature of the mammal, which may be from about 30 to about 40° C. in some embodiments, such as for certain domesticated pets (e.g., dog or cat) or livestock (e.g., cow, horse, sheep, or pig). Generally, the Tt is higher than the storage conditions of the formulation (which may be from about 2° C. to about 30° C., or about 10° C. to about 25° C., or from about 15° C. to about 22° C., or about 2° C. to about 8° C.), such that the therapeutic agent remains in solution for injection. Alternatively, the therapeutic agent may be stored frozen, such as from about −80° C. to about −20° C.

In some embodiments, the ELP can provide a transition temperature at a range of 27° C. to 36° C. inclusive. In some embodiments, the ELP can provide a transition temperature at a range of 28° C. to 35° C. inclusive. In some embodiments, the ELP can provide a transition temperature at a range of 29° C. to 34° C. inclusive. In some embodiments, the ELP can provide a transition temperature at a range of 27° C. to 33° C. inclusive. In some embodiments, the ELP can provide a transition temperature at a range of 30° C. to 33° C. inclusive. In some embodiments, the ELP can provide a transition temperature at a range of 31° C. to 31° C. inclusive. In some embodiments, the ELP can provide a transition temperature of 27° C., 28° C., 29° C., 30° C., 31° C., 32° C., 33° C., 34° C., 35° C., or 36° C. In some embodiments, the ELP can provide a transition temperature at a range of 28° C. to 35° C. inclusive at a protein concentration of 10 mg/mL in 110 mM NaCl.

In some embodiments, the ELP protein polymers are constructed through recursive ligation to rapidly clone DNA encoding highly repetitive polypeptides of any sequence and specified length over a large range of molecular weights. In a single cycle, two halves of a parent plasmid, each containing a copy of an oligomer, are ligated together, thereby dimerizing the oligomer and reconstituting a functional plasmid. This process is carried out recursively to assemble an oligomeric gene with the desired number of repeats. For example, one ELP structural subunit (e.g. a pentapeptide or a 9-mer of pentapeptides) is inserted into a vector. The vector is digested, and another ELP structural unit (e.g. a pentapeptide or a 9-mer of pentapeptides) is inserted. Each subsequent round of digestion and ligation doubles the number of ELP structural units contained in the resulting vector until the ELP polymer is the desired length. By varying the number of pentapeptides in the initial structural unit, ELPs of varying length can easily be constructed. Alternative means of construction (i.e. other than recursive ligation) can be used to produce alternative lengths of ELP.

In some embodiments, the vector contains one or more additional amino acids or ELP structural unit repeats. For example, the vector may add an additional pentamer repeat to the N terminus of the ELP with valine in the guest position and an additional pentamer to the C terminus with a tryptophan in the guest residue. The tryptophan may be used as a means to increase the extinction coefficient of the molecule, allowing for better measurement of absorbance, for instance at 280 nm, which can be useful for determination of protein concentration, or for monitoring protein content during purification. The pentamers added to either end can also be designed so as the encoding DNA contains restriction enzyme recognition sites for cloning of fusion partners on to either end of the ELP coding sequence.

### Methods of Treatment

In some aspects, the present disclosure provides a method of treating inflammatory lung damage (e.g. caused by COVID-19, ARDS), symptoms, and/or related conditions comprising administering pharmaceutical compositions of a vasoactive intestinal peptide and one or more ELPs to a subject in need. In some embodiments, the symptom and/or related condition is respiratory distress and/or failure. In some embodiments, respiratory failure is defined based on resource utilization requiring at least one of the following: endotracheal intubation and mechanical ventilation, oxygen delivered by high-flow nasal cannula; heated, humidified, oxygen delivered via reinforced nasal cannula at flow rates>20 L/min with fraction of delivered oxygen≥0.5, noninvasive positive pressure ventilation, extracorporeal membrane oxygenation (ECMO), and/or clinical diagnosis of respiratory failure (e.g. clinical need for one of the preceding therapies, but preceding therapies not able to be administered in settings of resource limitation).

In some aspects, the present disclosure provides a method of treating inflammatory lung damage in a subject in need thereof comprising administering pharmaceutical compositions of a vasoactive intestinal peptide and one or more ELPs to the subject. In some aspects, the present disclosure provides a method of preventing inflammatory lung damage in a subject in need thereof comprising administering pharmaceutical compositions of a vasoactive intestinal peptide and one or more ELPs to the subject. In some aspects, the present disclosure provides a method of slowing the progression of inflammatory lung damage in a subject in need thereof comprising administering pharmaceutical compositions of a vasoactive intestinal peptide and one or more ELPs to the subject. In some aspects, the present disclosure provides a method of ameliorating the symptoms of inflammatory lung damage in a subject in need thereof comprising administering pharmaceutical compositions of a vasoactive intestinal peptide and one or more ELPs to the subject.

In some embodiments, the inflammatory lung damage is associated with and/or caused by viral infection (e.g. SARS, COVID-19, MERS, coronavirus infections, or influenza infections), bacterial infection, pneumonia, interstitial lung disease, interstitial pneumonia, idiopathic pulmonary fibrosis, nonspecific interstitial pneumonitis, hypersensitivity pneumonitis, cryptogenic organizing pneumonia (COP), acute interstitial pneumonitis, sarcoidosis, pulmonary lung disease, inflammatory pulmonary disease, cytokine mediated lung injury, cytokine mediated acute lung injury, acute respiratory distress syndrome (ARDS), sepsis-related ARDS, pulmonary inflammation, or acute lung injury (ALI).

In some embodiments, the present disclosure provides a method of decreasing inflammation in a subject in need thereof comprising administering pharmaceutical compositions of a vasoactive intestinal peptide and one or more ELPs to the subject. In some embodiments, the present disclosure provides a method of decreasing fibrosis in a subject in need thereof comprising administering pharmaceutical compositions of a vasoactive intestinal peptide and one or more ELPs to the subject. In some embodiments, the present disclosure provides a method of decreasing fibroblast formation in a subject in need thereof comprising administering pharmaceutical compositions of a vasoactive intestinal peptide and one or more ELPs to the subject. In some embodiments, the present disclosure provides a method of decreasing expression of markers of inflammation or fibrosis in the lung in a subject in need thereof comprising administering pharmaceutical compositions of a vasoactive intestinal peptide and one or more ELPs to the subject. In some embodiments, the present disclosure provides a method of decreasing expression of one or more pro-inflammatory cytokines in a subject in need thereof comprising administering pharmaceutical compositions of a vasoactive intestinal peptide and one or more ELPs to the subject. In some embodiments, the one or more pro-inflammatory cytokines is implicated in the inflammatory response to lung injury. In some embodiments, the one or more pro-inflammatory cytokines include, but are not limited to, IL-17A, TNFα, NF-κβ, NLRP3, IL-18, IL-1, IL-1R TNTαR, and IL-18R.

In some embodiments, the present disclosure provides a method of increasing the expression of one or more anti-inflammatory cytokines in a subject in need thereof comprising administering pharmaceutical compositions of a vasoactive intestinal peptide and one or more ELPs to the subject. In some embodiments, the one or more anti-inflammatory cytokines include, but are not limited to the receptors that bind to one or more pro-inflammatory cytokines, interleukin-1 receptor antagonist, IL-4, IL-6, IL-10, IL-11, and IL-13. In some embodiments, the present disclosure provides a method of inducing the proliferation of protective T-cells in a subject in need thereof comprising administering pharmaceutical compositions of a vasoactive intestinal peptide and one or more ELPs to the subject.

The treatment, prevention, delay, or amelioration of inflammatory lung damage and/or one or more symptoms thereof may be measured by any means known in the art. For example, the treatment, prevention, delay, or amelioration of inflammatory lung damage and/or the symptoms thereof may be measured by assessing changes in oxygen levels in the patient, exhaled nitric oxide respiratory rate, or spirometry measurements. In some embodiments, inflammatory lung damage and/or one or more symptoms thereof is treated, prevented, delayed, or ameliorated for about 1 week, about 1 month, about 2 months, about 3 months, about 4 months, about 5 months, about 6 months, about 8 months, about 1 year, about 2 years, about 5 years, and/or about 10 years. In some embodiments, inflammatory lung damage and/or one or more symptoms thereof is prevented, delayed, or ameliorated by about 1%, about 5%, about 10%, about 20%, about 30%, about 40%, about 50%, about 60%, about 70%, about 80%, or about 90% compared with that in an untreated inflammatory lung damage patient or the same patient before treatment. In some embodiments, this prevention, delay, or amelioration of inflammatory lung damage and/or one or more symptoms thereof is observed at the time points disclosed herein.

In some embodiments, administration of the pharmaceutical compositions disclosed herein reduce inflammatory lung damage and/or improve one or more symptoms thereof in a subject compared to an untreated inflammatory lung damage subject or the same patient before treatment. In some embodiments, inflammatory lung damage is reduced and/or one or more symptoms thereof are improved for about 1 week, about 1 month, about 2 months, about 3 months, about 4 months, about 5 months, about 6 months, about 8 months, about 1 year, about 2 years, about 5 years, and/or about 10 years compared to an untreated inflammatory lung damage subject or the same patient before treatment. In some embodiments, inflammatory lung damage is reduced and/or one or more symptoms thereof is improved by about 1%, about 5%, about 10%, about 20%, about 30%, about 40%, about 50%, about 60%, about 70%, about 80%, or about 90% compared with an untreated inflammatory lung damage subject or the same patient before treatment. In some embodiments, this reduction in inflammatory lung damage and/or improvement in one or more symptoms thereof is observed at the time points disclosed herein.

In some embodiments, administration of the pharmaceutical compositions disclosed herein improves cardiac function in a subject compared to an untreated subject or the same patient before treatment. In some embodiments, cardiac function is improved for about 1 week, about 1 month, about 2 months, about 3 months, about 4 months, about 5 months, about 6 months, about 8 months, about 1 year, about 2 years, about 5 years, and/or about 10 years compared to an untreated subject or the same patient before treatment. In some embodiments, cardiac function is improved by about 1%, about 5%, about 10%, about 20%, about 30%, about 40%, about 50%, about 60%, about 70%, about 80%, or about 90% compared with an untreated subject or the same patient before treatment.

In some embodiments, this improvement in cardiac function is observed at the time points disclosed herein.

### Pharmaceutical Compositions and Administration

The present disclosure provides pharmaceutical compositions including a Vasoactive Intestinal Peptide and one or more ELPs with one or more pharmaceutically acceptable excipients and/or diluents. In some embodiments, the Vasoactive Intestinal Peptide and one or more ELPs is PB1046.

The present disclosure provides sustained release formulations including a therapeutic agent disclosed herein and one or more pharmaceutically acceptable excipients and/or diluents. For example, such excipients include salts, and other excipients that may act to stabilize hydrogen bonding. Any appropriate excipient known in the art may be used. Exemplary excipients include, but are not limited to, amino acids such as histidine, glycine, or arginine; glycerol; sugars, such as sucrose; surface active agents such as polysorbate 20 and polysorbate 80; citric acid; sodium citrate; antioxidants; salts including alkaline earth metal salts such as sodium, potassium, and calcium; counter ions such as chloride and phosphate; preservatives; sugar alcohols (e.g. mannitol, sorbitol); and buffering agents. Exemplary salts include sodium chloride, potassium chloride, magnesium chloride, calcium chloride, sodium phosphate dibasic, sodium phosphate monobasic, potassium phosphate monobasic, and potassium phosphate dibasic. In certain embodiments, the pharmaceutical compositions disclosed herein have enhanced efficacy, bioavailability, therapeutic half-life, persistence, degradation resistance, etc.

In certain embodiments, the formulation may include from about 5 mM histidine to about 100 mM histidine. In some embodiments, the formulation includes about 50 mM histidine, about 40 mM histidine, about 30 mM histidine, about 25 mM histidine, about 20 mM histidine, or about 15 mM histidine. In certain embodiments, the formulation may include from about 10 mM sodium chloride to about 200 mM sodium chloride. In some embodiments, the formulation includes about 20 mM sodium chloride, about 40 mM sodium chloride, about 60 mM sodium chloride, about 75 mM sodium chloride, about 100 mM sodium chloride, about 120 mM sodium chloride, or about 150 mM sodium chloride. In certain embodiments, the formulation may include from about 10 mM histidine to about 30 mM histidine and from about 60 mM sodium chloride to about 80 mM sodium chloride. In certain embodiments, the formulation may include about 20 mM histidine and about 75 mM sodium chloride.

The pharmaceutical composition is formulated at a pH, ionic strength, and generally with excipients sufficient to enable the formation of the matrix at body temperature (e.g., 37° C., or at from 34 to 36° C. in some embodiments). The pharmaceutical composition is generally prepared such that it does not form the matrix at storage conditions. The formulation can be stored frozen, refrigerated or at room temperature. The storage condition may be below freezing, such as lower than about −10° C., or lower than about −20° C., or lower than about −40° C., or lower than about −70° C. Storage conditions are generally less than the transition temperature of the formulation, such as less than about 32° C., or less than about 30° C., or less than about 27° C., or less than about 25° C., or less than about 20° C., or less than about 15° C. In some embodiments, the formulation is stored at 2°-8° C. For example, the formulation may be isotonic with blood or have an ionic strength that mimics physiological conditions. For example, the formulation may have an ionic strength of at least that of 25 mM Sodium Chloride, or at least that of 30 mM Sodium chloride, or at least that of 40 mM Sodium Chloride, or at least that of 50 mM Sodium Chloride, or at least that of 75 mM Sodium Chloride, or at least that of 100 mM Sodium Chloride, or at least that of 150 mM Sodium Chloride. In certain embodiments, the formulation has an ionic strength equivalent to that of 0.9% saline (154 mM sodium chloride).

In some embodiments, the formulation is formulated at physiological pH. In some embodiments, the formulation is formulated at a pH in the range of about 5.5 to about 8.5. In some embodiments, the formulation is formulated at a pH in the range of about 6.0 to about 8.0. In some embodiments, the formulation is formulated at a pH in the range of about 6.5 to about 7.5. In some embodiments, the formulation is formulated at a pH of 7.5. In some embodiments, formulations with a lower pH demonstrate improved formulation stability compared to formulations at a higher pH. In some embodiments, formulations with a higher pH demonstrate improved formulation stability compared to formulations at a lower pH.

In some embodiments, the formulation is stable at storage conditions. Stability can be measured using any appropriate means in the art. Generally, a stable formulation is one that shows less than a 5% increase in degradation products or impurities. In some embodiments, the formulation is stable for at least about 1 month, at least about 2 months, at least about 3 months, at least about 4 months, at least about 5 months, at least about 6 months, at least about one year, or at least about 2 years or more at the storage conditions. In some embodiments, the formulation is stable for at least about 1 month, at least about 2 months, at least about 3 months, at least about 4 months, at least about 5 months, at least about 6 months, or at least about one year or more at 25° C.

The protein concentration in the formulation is tailored to enable the formation of the coacervate at the temperature of administration. For example, higher protein concentrations help drive the formation of the coacervate, and the protein concentration needed for this purpose varies depending on the ELP series used. For example, in some embodiments using an ELP1-120, or ELPs with comparable transition temperatures, the protein is present in the range of about 1 mg/mL to about 200 mg/mL, or is present in the range of about 5 mg/mL to about 125 mg/mL.

In exemplary embodiments, the disclosure provides a sustained release pharmaceutical composition that includes a vasoactive intestinal peptide disclosed herein (e.g. having an N-terminal moiety such as a Methionine) and one or more amino acid sequences including [VPGXG]₉₀, [VPGXG]₁₂₀, [VPGXG]₁₆₀, or [VPGXG]₁₈₀ where each X is selected from V, G, and A. V, G, and A may be present at a ratio of about 5:3:2. The formulation further includes one or more pharmaceutically acceptable excipients and/or diluents for formation of a reversible matrix from an aqueous form upon administration to a human subject. VIP and derivatives thereof are disclosed in U.S. Patent Publication No. 2011/0178017.

In another aspect, the disclosure provides a method for delivering a sustained release regimen of a vasoactive intestinal peptide disclosed herein. The method comprises administering the pharmaceutical composition described herein to a subject in need, wherein the pharmaceutical composition is administered from about 1 to about 8 times per month. In some embodiments, the pharmaceutical composition is administered about 1 time, about 2 times, about 3 times, and/or about 4 times per month. In some embodiments, the pharmaceutical composition is administered weekly. In some embodiments, the pharmaceutical composition is administered daily. In some embodiments, the pharmaceutical composition is administered from one to three times weekly. In some embodiments, the pharmaceutical composition is administered once every two weeks. In some embodiments, the pharmaceutical composition is administered from one to two times a month. In particular embodiments, the pharmaceutical composition is administered about 1 time per month. In some embodiments, the pharmaceutical composition is administered about once every 2 months, about once every 3 months, about once every 4 months, about once every 5 months, and/or about once every 6 months. In some embodiments, VIP may have an additional moiety such as Methionine at the N-terminus to alter the receptor binding profile, as described in U.S. Patent Publication No. 2011/0178017. In some embodiments, VIP is fused to ELP1 (having from about 90 to about 180 ELP units).

The pharmaceutical composition can be packaged in the form of pre-filled pens or syringes for administration once per week, once every 7 days, twice per week, or from one to eight times per month, or alternatively filled in conventional vials and the like.

Advantageously, the compositions provide for prolonged pharmacokinetic exposure due to sustained release of the active agent. In particular aspects, the maximal exposure level may be achieved at about 10 hours, about 24 hours, about 48 hours, about 60 hours, or about 72 hours after administration; typically the maximum exposure level is achieved between about 10 hours and about 48 hours after administration. After the maximal exposure level is achieved the compositions may achieve a sustained level of release whereby a substantial percentage of the maximal level is obtained for a period of time. For example, the sustained level may about 50%, about 60%, about 70%, about 80%, about 90% or about 100%. Exemplary periods of time for maintaining the sustained rate are about 3 days, about 4 days, about 5 days, about 6 days, about 1 week, about 2 weeks, about 4 weeks, about 6 weeks, or about 8 weeks, after the maximal exposure rate is achieved. Subsequently, the sustained level may lower to a reduced exposure level. Such reduced exposure rates may be about 5%, about 10%, about 20%, about 30%, about 40%, about 50% or about 60%.

In some embodiments, the pharmaceutical compositions disclosed herein are administered chronically. In some embodiments, the pharmaceutical compositions disclosed herein are administered for about 6 months, for about 7 months, for about 8 months, for about 9 months, for about 10 months, for about 11 months, for about 1 year, for about 2 years, for about 3 years, for about 4 years, for about 5 years, for about 10 years or more. The pharmaceutical compositions may be administered at any required dose and/or frequency disclosed herein.

In some embodiments, the pharmaceutical compositions disclosed herein are administered until inflammatory lung damage is reduced and/or one or more symptoms thereof improve. In some embodiments, the pharmaceutical compositions disclosed herein are administered until inflammatory lung damage and/or one or more symptoms thereof is ameliorated. In some embodiments, the pharmaceutical compositions disclosed herein are administered until inflammatory lung damage and/or one or more symptoms thereof is delayed. In some embodiments, the pharmaceutical compositions disclosed herein are administered until inflammatory lung damage and/or one or more symptoms thereof is cured.

In some embodiments, the pharmaceutical compositions disclosed herein are administered before the patient begins to exhibit one or more inflammatory lung damage symptoms. In some embodiments, the pharmaceutical compositions disclosed herein are administered at the onset of inflammatory lung damage symptoms.

The pharmaceutical composition is generally for “systemic delivery,” meaning that the agent is not delivered locally to a pathological site or a site of action. Instead, the agent is absorbed into the bloodstream from the injection site, where the agent acts systemically or is transported to a site of action via the circulation. The therapeutic agent may be administered by any known route, such as for example, orally, intravenously, intramuscularly, nasally, subcutaneously, intra-vaginally, and intra-rectally. In one embodiment, the formulation is generally for subcutaneous administration. In one embodiment, the pharmacokinetic (PK) parameters are prolonged when the agent is administered subcutaneously. In one embodiment, the half-life of the fusion protein is prolonged. In one embodiment, the PK parameters when the agent is administered subcutaneously are prolonged compared with the agent administered by other means (e.g. intravenously). In one embodiment, the depot of the agent is prolonged when the agent is administered subcutaneously compared with the agent administered by other means (e.g. intravenously).

In some embodiments, the formulation is administered about monthly, and may be administered subcutaneously or intramuscularly. In some embodiments, the formulation is administered about weekly, and may be administered subcutaneously or intramuscularly. In some embodiments, the site of administration is not a pathological site, for example, is not the intended site of action.

In various embodiments, the plasma concentration of the active agent does not change by more than a factor of 10, or a factor of about 5, or a factor of about 3 over the course of a plurality of administrations, such as at least 2, at least about 5, or at least about 10 administrations of the formulation. The administrations are substantially evenly spaced, such as, for example, about daily, or about once per week, or from one to about five times per month, or about once every two months, or about once every three months.

The pharmaceutical compositions disclosed herein may be administered in smaller doses and/or less frequently than unfused or unconjugated counterparts.

While one of skill in the art can determine the desirable dose in each case, a suitable dose of the pharmaceutical composition for achievement of therapeutic benefit, may, for example, be in a range of about 1 microgram (μg) to about 100 milligrams (mg) per kilogram body weight of the recipient, preferably in a range of about 10 μg to about 50 mg per kilogram body weight and most preferably in a range of about 100 μg to about 10 mg per kilogram body weight. In some embodiments, the pharmaceutical composition is administered at a low dose. In some embodiments, the pharmaceutical composition is administered at a dose between 0.1 mg per kilogram per body weight to about 9 mg per kilogram per body weight. In some embodiments, the pharmaceutical composition is administered at about 0.05 mg per kilogram body weight, about 0.1 mg per kilogram body weight, about 0.2 mg per kilogram body weight, about 0.4 mg per kilogram body weight, about 0.5 mg per kilogram body weight, about 0.8 mg per kilogram body weight, about 1 mg per kilogram body weight, about 1.2 mg per kilogram body weight, about 2 mg per kilogram body weight, about 3 mg per kilogram body weight, and/or about 9 mg per kilogram body weight. The desired dose may be administered weekly.

A suitable dose of the pharmaceutical composition for achievement of therapeutic benefit, may, for example, be in a range of about 1 microgram (μg) to about 10 milligrams (mg) per kilogram body weight of the recipient, preferably in a range of about 10 μg to about 5 mg per kilogram body weight, and most preferably in a range of about 100 μg to about 2 mg per kilogram body weight. In some embodiments, the pharmaceutical composition is administered at a low dose. In some embodiments, the pharmaceutical composition is administered at a dose between 0.1 mg per kilogram per body weight to about 9 mg per kilogram per body weight. In some embodiments, the pharmaceutical composition is administered at about 0.05 mg per kilogram body weight, about 0.1 mg per kilogram body weight, about 0.2 mg per kilogram body weight, about 0.4 mg per kilogram body weight, about 0.5 mg per kilogram body weight, about 0.8 mg per kilogram body weight, about 1 mg per kilogram body weight, about 1.2 mg per kilogram body weight, about 2 mg per kilogram body weight, about 3 mg per kilogram body weight, and/or about 9 mg per kilogram body weight. The desired dose may be presented as one dose or two or more sub-doses administered at appropriate intervals. These sub-doses can be administered in unit dosage forms, for example, containing from about 10 μg to about 1000 mg, preferably from about 50 μg to about 500 mg, and most preferably from about 50 μg to about 250 mg of active ingredient per unit dosage form. Alternatively, if the condition of the recipient so requires, the doses may be administered as a continuous infusion.

In some embodiments, the pharmaceutical composition is administered in a fixed dose, regardless of the weight of the patient at a dose of about 1 mg to about 1 g or more. In some embodiments, the pharmaceutical composition is administered at a dose of about 1.0 mg, about 1.1 mg, about 1.2 mg, about 1.3 mg, about 1.4 mg, about 1.5 mg, about 1.6 mg, about 1.7 mg, about 1.8 mg, about 1.9 mg, about 2.0 mg, about 3.0 mg, about 4.0 mg, about 5.0 mg, about 6.0 mg, about 7.0 mg, about 8.0 mg, about 9.0 mg, about 10.0 mg, about 15 mg, about 20 mg, about 25 mg, about 30 mg, about 35 mg, about 40 mg, about 45 mg, about 50 mg, about 55 mg, about 60 mg, about 65 mg, about 70 mg, about 75 mg, about 80 mg, about 85 mg, about 90 mg, about 95 mg, about 100 mg, about 110 mg, about 120 mg, about 130 mg, about 140 mg, about 150 mg, about 160 mg, about 170 mg, about 180 mg, about 190 mg, about 200 mg, about 210 mg, about 220 mg, about 230 mg, about 240 mg, about 250 mg, about 260 mg, about 270 mg, about 280 mg, about 290 mg, about 300 mg, about 310 mg, about 320 mg, about 330 mg, about 340 mg, about 350 mg, about 360 mg, about 370 mg, about 380 mg, about 390 mg, about 400 mg, about 410 mg, about 420 mg, about 430 mg, about 440 mg, about 450 mg, about 460 mg, about 470 mg, about 480 mg, about 490 mg, about 500 mg, about 600 mg, about 700 mg, about 800 mg, about 900 mg, about 1 g or more. In some embodiments, the pharmaceutical composition is administered at a dose of about 10 mg, 40 mg, or 100 mg.

The pharmaceutical composition may be administered for between about one day to about one year or more. In some embodiments, the pharmaceutical composition is administered for one day, two days, three days, four days, five days, six days, seven days, two weeks, three weeks, four weeks, one month, two months, three months, four months, five months, six months, seven months, eight months, nine months, ten months, eleven months, twelve months or more.

The pharmaceutical composition of the present disclosure may be administered by any known route, such as intravenously, intramuscularly, nasally, subcutaneously, intra-vaginally, intra-rectally, and the like; and the therapeutic agent may also be administered by any conventional route. In many embodiments, at least one therapeutic agent may be administered subcutaneously.

In some embodiments, the pharmaceutical composition is subcutaneously administered to the subject at a dose of about 10 mg per week for 4 weeks or until hospital discharge. In some embodiments, the pharmaceutical composition is subcutaneously administered to the subject at a dose of about 40 mg per week for 4 weeks or until hospital discharge. In some embodiments, the pharmaceutical composition is subcutaneously administered to the subject at a dose of about 100 mg per week for 4 weeks or until hospital discharge.

In certain embodiments, the subject is a human, but in other embodiments may be a non-human mammal, such as a domesticated pet (e.g., dog or cat), or livestock or farm animal (e.g., horse, cow, sheep, or pig).

### Patient Population

Any appropriate patient may be administered the pharmaceutical compositions of the present disclosure. In some embodiments, the patient is at risk of developing inflammatory lung damage. In some embodiments, the patient is experiencing inflammatory lung damage. In some embodiments, the patient has developed inflammatory lung damage. In some embodiments, the patient is infected with an agent that is associated with and/or causes inflammatory lung damage. In some embodiments, the patient is infected with a virus that is associated with and/or causes inflammatory lung damage. In some embodiments, the virus that is associated with and/or causes inflammatory lung damage is a coronavirus or an influenza virus. In some embodiments, the coronavirus is MERS-CoV, SARS, SARS-CoV-1 or SARS-CoV-2. In some embodiments, the patient is infected with, or presumed to be infected with, SARS-CoV-2. In some embodiments, the patient has COVID-19, ARDS, and/or is experiencing symptoms associated with COVID-19 or ARDS. In some embodiments, the patient is hospitalized. In some embodiments, the patient has a mild case of COVID-19, ARDS, and/or is experiencing mild symptoms associated with COVID-19 or ARDS. In some embodiments, the patient has a moderate case of COVID-19, ARDS, and/or is experiencing moderate symptoms associated with COVID-19 or ARDS. In some embodiments, the patient has a severe case of COVID-19, ARDS, and/or is experiencing severe symptoms associated with COVID-19 (e.g. rapid clinical deterioration, ARDS, and/or death). In some embodiments, the patient requires assistance breathing. In some embodiments, the patient is receiving supplemental oxygen. In some embodiments, the patient is receiving supplemental oxygen by face mask or nasal cannula with prongs. In some embodiments, the patient requires a ventilator to breathe. In some embodiments, the patient exhibits low oxygen saturation levels. In some embodiments, the patient exhibits an increased respiratory rate (e.g. greater than 24 breaths/minute). In some embodiments, the patient exhibits an accompanying fever (e.g. temperature greater than 100.4° F. or 38° C.). In some embodiments, the patient is at risk of progressing to more severe COVID-19,ARDS, or symptoms thereof, and the pharmaceutical composition of the present disclosure is administered before symptoms worsen.

In some embodiments, the patient is determined as having mild COVID-19 by 1) positive testing by standard RT-PCR assay or the equivalent; 2) symptoms of mild illness with COVID-19 that could include fever, cough, sore throat, malaise, headache, muscle pain, gastrointestinal symptoms, without shortness of breath of dyspneas; and/or 3) no clinical signs indicative of moderate, severe, or critical COVID-19.

In some embodiments, the patient is determined as having moderate COVID-19 by 1) positive testing by standard RT-PCR assay or the equivalent; 2) symptoms of moderate illness with COVID-19, which can include any symptom of mild illness or shortness of breath with exertion; 3) clinical signs suggestive of moderate illness with COVID-19, such as respiratory rate≥20 breaths per minute, saturation of oxygen (SpO₂)>93% on room air at sea level, heart rate≥90 beats per minute; and/or 4) no clinical signs indicative of severe or critical illness.

In some embodiments, the patient is determined as having severe COVID-19 by 1) positive testing by standard RT-PCR assay or the equivalent; 2) symptoms suggestive of severe systemic illness with COVID-19, which can include any symptom of moderate illness or shortness of breath at rest, or respiratory distress; 3) clinical signs indicative of severe systemic illness with COVID-19, such as respiratory rate≥30 per minute, heart rate≥125 per minute, SpO₂≤93% on room air at sea level, or PaO₂/FiO₂<300 and/or 4) no criteria for critical severity.

In some embodiments, the patient is determined as having severe COVID-19 by 1) positive testing by standard RT-PCR assay or the equivalent; 2) evidence of critical illness, defined by at least one of the following: a) respiratory failure defined based on resource utilization requiring at least one of the following: endotracheal intubation and mechanical ventilation, oxygen delivered by high-flow nasal cannula; heated, humidified, oxygen delivered via reinforced nasal cannula at flow rates>20 L/min with fraction of delivered oxygen≥0.5, noninvasive positive pressure ventilation, extracorporeal membrane oxygenation (ECMO), and/or clinical diagnosis of respiratory failure (e.g. clinical need for one of the preceding therapies, but preceding therapies not able to be administered in settings of resource limitation); b) shock (e.g. defined by systolic blood pressure<90 mmHg, or diastolic blood pressure<60 mmHg, or requiring vasopressors); and/or c) multi-organ dysfunction/failure.

In some embodiments, the patient is an infant (e.g. 0 to 2 years old inclusive). In some embodiments, the patient is a pediatric patient (e.g. 2 to 18 years old inclusive). In some embodiments the patient is between the ages of 18 and 100. In some embodiments, the patient is between the ages of 18 and 85. In some embodiments, the patient is between the ages of 50 and 100. In some embodiments, the patient is older than 65 years old. In some embodiments, the patient is 60, 61, 62, 63, 64, 65, 66, 67, 68, 69, 70 ,71, 72, 73, 74, 75, 76, 77, 78, 79, 80, 81, 82, 83, 84, 85, 86, 87, 88, 89, 90, 91, 92, 93, 94, 95, 96, 97, 98, 99, or 100 years old or older. In some embodiments, the patient who is older than 50 years old is considered high risk of developing severe disease (e.g. COVID-19 or ARDS). In some embodiments, the patient who is older than 65 years old is considered high risk of developing severe disease (e.g. COVID-19 or ARDS).

In some embodiments, a patient who possess one or more comorbidities is considered high risk for developing severe disease (e.g. COVID-19 or ARDS). In some embodiments, the comorbidity includes, but is not limited to, obesity, hypertension, diabetes, an autoimmune disorder (e.g. rheumatoid arthritis), heart disease, heart failure, atherosclerosis, cancer (e.g. lung cancer), exposure to lung-damaging agents, liver disease, alcoholism, other pulmonary infection, and chronic kidney disease. In some embodiments, the patient at risk presents with elevated markers of cardiac injury or dysfunction (e.g. hsTnI, NT-proBNP). In some embodiments, race is a factor in a patient being considered at high risk of developing severe disease (e.g. COVID-19 or ARDS). In some embodiments, socioeconomic status is a factor in a patient being considered at high risk of developing severe disease (e.g. COVID-19 or ARDS).

In some embodiments, while being administered with a pharmaceutical composition of the present disclosure, the patient continues to receive standard of care for any comorbidities.

### Combination Therapies

The pharmaceutical compositions disclosed herein may be administered with various therapies used to treat, prevent, delay, or ameliorate inflammatory lung damage, COVID-19, and/or ARDS. In some embodiments, the pharmaceutical composition is administered concomitantly with standard of care medications. The one or more therapeutic agents may be any compound, molecule, or substance that exerts therapeutic effect to a subject in need thereof.

In some embodiments, the pharmaceutical compositions disclosed herein are administered with therapeutic agents including, but not limited to, antiviral agents, anti-malarial agents, agents that protect epithelial cells, defibrotide, convalescent plasma, chloroquine, hydroxychloroquine, remdesivir, desferal, favipiravir, corticosteroids, clevudine, anti-inflammatory agents, anti-oxidant agents, dapagliflozin, IFX-1, ruxolitinib, baricitinib, interferon beta 1a, azithromycin, tocilizumab, acalabrutinib, umifenovir, ciclesonide, sarilumab, anti-interleukin agents, and telmisartan.

The one or more therapeutic agents may be “co-administered”, i.e., administered together in a coordinated fashion to a subject, either as separate pharmaceutical compositions or admixed in a single pharmaceutical composition. By “co-administered”, the one or more therapeutic agents may also be administered simultaneously with the present pharmaceutical compositions, or be administered separately, including at different times and with different frequencies. The one or more therapeutic agents may be administered by any known route, such as orally, intravenously, intramuscularly, nasally, via aerosol, subcutaneously, intra-vaginally, intra-rectally, and the like; and the therapeutic agent may also be administered by any conventional route. In some embodiments, the pharmaceutical composition is administered subcutaneously.

When two or more therapeutic agents are used in combination, the dosage of each therapeutic agent is commonly identical to the dosage of the agent when used independently. However, when a therapeutic agent interferes with the metabolism of others, the dosage of each therapeutic agent is properly adjusted. Alternatively, where the two or more therapeutic agents show synergistic effects, the dose of one or more may be reduced. Each therapeutic agent may be administered simultaneously or separately in an appropriate time interval.

It should be understood that singular forms such as “a,” “an,” and “the” are used throughout this application for convenience, however, except where context or an explicit statement indicates otherwise, the singular forms are intended to include the plural. All numerical ranges should be understood to include each and every numerical point within the numerical range, and should be interpreted as reciting each and every numerical point individually. The endpoints of all ranges directed to the same component or property are inclusive, and intended to be independently combinable.

The term “about” when used in connection with a referenced numeric indication means the referenced numeric indication plus or minus up to 10% of that referenced numeric indication. For example, the language “about 50” covers the range of 45 to 55.

As used herein, the word “include,” and its variants, is intended to be non-limiting, such that recitation of items in a list is not to the exclusion of other like items that may also be useful in the materials, compositions, devices, and methods of this technology. Similarly, the terms “can” and “may” and their variants are intended to be non-limiting, such that recitation that an embodiment can or may comprise certain elements or features does not exclude other embodiments of the present technology that do not contain those elements or features. Although the open-ended term “comprising,” as a synonym of terms such as including, containing, or having, is used herein to describe and claim the disclosure, the present technology, or embodiments thereof, may alternatively be described using more limiting terms such as “consisting of” or “consisting essentially of” the recited ingredients.

Unless defined otherwise, all technical and scientific terms herein have the same meaning as commonly understood by one of ordinary skill in the art to which this disclosure belongs. Although any methods and materials, similar or equivalent to those described herein, can be used in the practice or testing of the present disclosure, the preferred methods and materials are described herein.

This disclosure is further illustrated by the following non-limiting examples.

### EXAMPLES

### Example 1—Dosing Strategry for PB1046

The single and multiple-dose PK profiles of PB1046 administered SC demonstrated a dose-related but less than dose-proportional increase in exposure with the expected prolonged release/absorption kinetics of ELP-based compounds that could support a weekly dosing regimen.

In a multiple ascending dose study in which weekly SC injections of PB1046 was administered for 4 weeks in patients with heart failure added to their standard of care heart failure regimen, the PK profile of PB1046 demonstrated dose-dependent but less than dose-proportional increases in mean serum drug concentrations. Dose levels in each of the dose cohorts 1, 2, 3 and 4 were 0.2, 0.4, 0.6 and 1.2 mg/kg respectively in study NCT02808585. Based on non-compartmental PK analysis and dose-exposure-related covariate analysis, body weight and BMI were not considered significant covariates.

### Example 2—Clinical Trial to Assess the Efficacy and Safety of Once Weekly Subcutaneous Injections of PB1046 in Hospitalized COVID-19 Patients at High Risk for Rapid Clinical Deterioration and ARDS

The target indication for PB1046 is to provide significant clinical improvement to hospitalized COVID-19 patients at high risk for rapid clinical deterioration, ARDS, and death.

PB1046 Subcutaneous Injection is provided as a clear, colorless to slightly yellow liquid at either 40 mg/mL or 100 mg/mL in 2 mL or 3 mL clear glass single use vials. Both concentrations of PB1046 are formulated in 20 mM histidine buffer, 75 mM NaCl at near neutral pH.

Vials will be stored long term at≤−70° C. Short term storage at −20° C. (±10° C.) or refrigerated (2-8° C.) temperatures may be permitted based on ongoing stability analysis, however, refrigerated storage must be discussed with the Sponsor in advance. Refer to Certificate of Analysis (COA) for additional drug storage stability information.

### Study Rationale

There are no effective therapies for management of COVID-19 patients who are at high risk of rapid clinical deterioration and death. Treatment options are mostly supportive and non-specific. The immunomodulatory effects of VIP have been shown in animal models and human clinical trials to be effective in reducing lung injury and pulmonary complications in conditions similar to COVID-19. Here the potential therapeutic benefit of PB1046 to accelerate clinical improvement in hospitalized COVID-19 patients who are highly symptomatic and at high risk of rapid clinical deterioration, ARDS, and death will be investigated.

### Dosing Rationale

The dosing strategy is based on earlier results disclosed in Example 1. For the current study, the 0.2, 0.4, and 1.2 mg/kg dose levels tested previously were converted to fixed doses of 10 mg, 40 mg, and 100 mg, respectively, based on expected mean body weights and BMI's expected in the study population. Because no significant PB1046 pharmacodynamic effect was observed for the 0.2 mg/kg (10 mg) dose level, this low dose will be considered the control arm for the study. The injection site reactions observed previously at the 0.2 mg/kg dose level supports maintenance of study blinding during the active treatment period of the study.

### Study Design

This is a multicenter, randomized, double-blind, parallel group study to investigate the efficacy of weekly, subcutaneously administered PB1046 in reducing the time to discharge of COVID-19 patients at high risk for clinical deterioration. Clinical improvement will be assessed with time to discharge evaluated at 28 days, time to clinical recovery and an 8-category ordinal scale of clinical improvement recommended by the World Health Organization (WHO) for clinical trials investigating therapeutics for COVID-19 patients will also be assessed. Other clinical parameters will also be investigated, such as, the need for ICU admission, mechanical ventilation, development of ARDS, time to discharge, days alive and out of hospital and overall mortality. Importantly, the change in cardiac markers of injury and dysfunction, including high sensitivity troponin I (hs-TrI) and NT-proBNP, as well as change in known inflammatory markers of COVID-19 disease progression, such as TNF-alpha, IL-1, and IL-6, will also be examined. The overall safety, tolerability, immunogenicity, and PK profiles of PB1046 in COVID-19 patients will also be assessed.

Study drug will be administered as a weekly subcutaneous (SC) injections initiated upon enrollment and continued once weekly until hospital discharge or for a maximum of 4 weeks during the hospitalization.

The study will enroll at least approximately 180 hospitalized COVID-19 patients at high risk of rapid clinical deterioration as described in the eligibility criteria. During screening, eligible patients must meet all of the eligibility criteria and provide written or witnessed verbal informed consent. Remote legal authorized representative (LAR) or remote family member as permitted by governing local or central Institutional Review Board (IRB)/Independent Ethics Committee (IEC) is permitted. Ordinal scale assessments should be recorded at baseline upon randomization and at least daily based on locally acquired data in the medical record, flow charts, weekly PB1046 dosing visits, and any other locally available documentation of patient status. All other baseline and post-treatment clinical parameters collected for the study, including vital signs, physical examinations, laboratory and imaging assessments, non-invasive or invasive ventilation parameters, respiratory care treatments or maneuvers, and any other clinical assessments may be obtained from the local medical record.

Upon enrollment, subjects will be randomized in a 1:1:1 ratio to 1 of 3 dose groups (low-, middle, or high-dose), with each dose group comprised of at least approximately 60 study subjects.

Low: PB1046 10 mg SC weekly×4 weeks or until hospital discharge

Middle: PB1046 40 mg SC weekly×4 weeks or until hospital discharge

High: PB1046 100 mg SC weekly×4 weeks or until hospital discharge

Upon randomization, all subjects will receive weekly SC injections of PB1046 for 4 weeks or until hospital discharge whichever is shorter. To minimize healthcare personnel and study staff exposure to COVID-19 and to minimize use of person protective equipment (PPE), all scheduled study assessments will occur only at the time of dose administration (once weekly) or collected from standard-of-care local assessments recorded during the hospitalization. To support assessment of the primary and other endpoints, the date and time of ordinal scale assessments should be recorded daily. The date, time, and indication for ICU (or palliative care) admissions and mechanical ventilation must be recorded, as well as date, time, and criteria for ARDS diagnosis (PaO2:FiO2 ratio), and date and time of mortality if applicable. To support assessment of other endpoints, all locally obtained laboratory results, imaging assessments, and oxygenation/ventilator settings occurring between study visits should be recorded daily and entered into source documents for subsequent eCRF input.

Safety monitoring of all treatment emergent adverse events (TEAEs), including serious adverse events (SAEs) and mortality, will occur for 35 days following initiation of PB1046. After hospital discharge, follow-up study visits may occur as remote video or telephone assessments or as in-person assessments in an out-patient or in-patient facility. Presence of anti-drug antibodies (ADA) will be assessed prior to PB1046 administration and at 7- and 35-days post-initiation of the PB1046 injections. If ADA's are still present at 35 days, the subject will be asked to provide an additional blood sample for ADA assessment 3 and/or 6 months later to assess resolution. A detailed schedule of study activities is shown in the Schedule of Events (SOE).

Although the risk of symptomatic hypotension with PB1046 has been minimal in the clinical development program to date, precautions should be taken in patients who are at high risk of hypotension or become overtly hypotensive during treatment. If hypotension occurs after treatment with PB1046, general supportive measures should be employed such as fluid boluses or use of vasopressor agents. In a preclinical study, sympathomimetic agents were shown to counteract the vasodilatory effects of PB1046.

### Patient Population

This study will recruit hospitalized COVID-19 patients at high risk for rapid clinical deterioration, ARDS, and death, who require urgent decision-making and treatment. Enrollment activities are streamlined to enable rapid treatment of study subjects. Written or witnessed verbal informed consent may be provided by the patient or provided in person or remotely by the patient's legally authorized representative (LAR), or family member as per local regulations. Approximately 200 subjects may be screened to meet the target enrollment number of 180 subjects.

### Inclusion Criteria

High-risk COVID-19 patients will be eligible for inclusion into the study if they meet all of the following criteria: Written or witnessed verbal informed consent from patient or remote legal authorized representative (LAR) or remote family member as permitted by governing local or central Institutional Review Board (IRB); Male or female 18-85 years old hospitalized COVID-19 patients (positive local SARS-CoV2 test); Receiving oxygen (O₂) by face mask or nasal cannula/prongs and/or with elevated markers of cardiac injury or dysfunction (hsTnI or NT-proBNP) as assessed by local testing.

Exclusion Criteria: Subjects will be excluded from the study if they meet any of the following criteria: Patients considered unsalvageable or expected to expire with 24 hours; On mechanical ventilation or imminent need for mechanical ventilation expected in the next 24 hours; Evidence of acute end-organ injury in 2 or more organ systems (not including cardiac or pulmonary), such as, renal, hepatic, gastrointestinal, or CNS injury; Receiving another investigational therapy for treatment or prevention of COVID-19-related hypoxemic respiratory failure or ARDS other than antiviral therapy; Systolic blood pressure (SBP)<95 mmHg and/or diastolic blood pressure (DBP)<50 mmHg or overt symptomatic hypotension during screening; Resting heart rate>110 BPM during screening; Severe chronic renal failure as measured by the estimated glomerular filtration rate (eGFR) of<30 mL/min/1.73 m² using the local laboratory calculation of eGFR. Significant liver dysfunction as measured by any one of the following at screening: ALT>3.0 times upper limit of normal (ULN); AST>3.0 times ULN; Serum bilirubin≥1.6 mg/dL; Any in-patient surgical procedure or hospitalization (defined as>23 hours) within 30 days of subject screening except for prior hospitalization for COVID-19; Known hypersensitivity to study drug or any of the excipients of the drug formulation; Pregnant or lactating female subjects; Any other condition which, in the opinion of the Investigator, would place the subject at increased risk or would preclude obtaining informed consent or confound the objectives of study.

This Phase 2 study will consist of:

Day 0 screening/pre-treatment period—Visit 1

Day 0 on-site randomization to study treatment and administration—Visit 2

Day 7 on-site study treatment and assessment visit (if not discharged)—Visit 3

Day 14 on-site treatment and assessment visit (if not discharged)—Visit 4

Day 21 on-site treatment and assessment visit (if not discharged)—Visit 5

Day 35+7 final follow-up visit, end of study (EOS)—Visit 6

Serum samples for PK analysis of PB1046 will be collected at screening/pre-treatment (Visit 1), on the 2nd, 3rd, and 4th treatment days prior to study drug administration (Visits 3, 4, 5, respectively), and at EOS Visit 6 if available. The existence of anti-drug-antibodies will be assessed in all subjects on Days 1 (pre-dose) and 35+7 (if available) following administration of study drug. Safety and tolerability will be carefully monitored throughout the study.

Patients will be enrolled in the study as soon as informed consent has been given and inclusion and exclusion criteria have been satisfied. The duration of hospitalization for each patient will be determined by clinical status independent of study procedures. The estimated duration of the study for each subject, including screening, is approximately 35+7 days. The subjects may be involved up to 42 days.

All subjects will be randomized to either a low control (10 mg), middle (40 mg), or high (100 mg) dose of active treatment. If subject is not discharged, they will continue to Day 7, 14, and 21 treatments. PB1046 is expected to improve the clinical outcomes of hospitalized to COVID-19 subjects with longer survival free from respiratory failure at 28 days.

The duration of hospitalization for each subject will be determined by clinical status independent of study procedures. The estimated duration of the study for each subject, including screening, is approximately 35+7 days. The subjects may be involved up to 42 days.

### Objectives and Endpoints

This is randomized, double-blind, parallel group study to investigate the therapeutic efficacy of high and middle dose levels of PB1046 compared to a low-dose level in hospitalized COVID-19 patients.

### Primary Objectives and Endpoints

Assess the effect of weekly, in-hospital subcutaneous administration of PB1046 on clinical improvement of hospitalized COVID-19 patients at high risk for clinical deterioration, ARDS, and death. The primary analysis will be based on those subjects enrolled in order to 113 discharges between high and low dose group. For assessment of the primary endpoint, a sample size of 70 subjects in each of the 3 dose groups will provide 80% power to detect to detect a 0.59 hazard ratio favoring high or medium dose group at the 5% significance level.

Primary Endpoint: Time to discharge [28 days].

Primary Endpoint Justification: The primary endpoint is a composite measure of clinical improvement and/or survival assessed at 28 days from initiation of PB1046 (post randomization). The ordinal scale measures illness severity over time. The primary outcome is a measure of a patient's improvement in clinical status over time. Use of a standardized clinical improvement scale supports agreement and consistency in recording of individual outcome events across the study population that will be enrolled at multiple centers and will facilitate interpretation and robustness of study results.

Alternative Primary Endpoint: proportion of patients alive and free of respiratory failure (e.g., need for non-invasive mechanical ventilation, high flow oxygen, or ECMO) at 28 days.

Second Alternative Primary Endpoint: days alive and removed from level of care (e.g. hospitalization, need for mechanical ventilation, etc.) at 28 days.

### Secondary Objectives and Endpoints

Secondary Objectives: To assess the effect of PB1046 on time to clinical recovery; To assess the effect of PB1046 on all-cause mortality; To assess the effect of PB1046 on development of ARDS; To assess the effect of PB1046 on overall hospital resource utilization; To assess the effect of PB1046 on need for admission to ICU; To assess the effect of PB1046 on need for mechanical ventilation (or palliative care); To assess the effect of PB1046 on the severity of respiratory failure as measured by the PaO2:FiO2 ratio; To assess the effect of PB1046 on need for high-flow oxygen or non-invasive ventilation; To assess the effect of PB1046 administration on markers of cardiac injury and cardiac dysfunction; To assess the effect of weekly PB1046 on markers of inflammation, such as TNF-alpha, IL-1, and IL-6; and To assess the overall safety and tolerability of PB1046.

Secondary Endpoints: Time to clinical recovery (being well enough for hospital discharge or returning to normal baseline activity level prior to discharge) [28 days]; All-cause mortality [28 days]; incidence of ARDS (PaO2:FiO2 ratio<300 mm Hg) during hospitalization; Reduction in hospital resource utilization defined by each of the following and as a composite; Total hospital days; Total ICU days; Total days of ventilator use; Total days of ECMO; Total days of invasive hemodynamic monitoring; Total days of mechanical circulatory support; Total days of inotropic or vasopressor therapy; Proportion of patients admitted to the intensive care unit (ICU); Proportion of patients requiring mechanical ventilation (or palliative care) due to hypoxic respiratory failure; Change in PaO2:FiO2 ratio in ventilated patients and patients with ARDS; Proportion of patients requiring high-flow oxygen therapy or non-invasive ventilation; Time to clinical improvement as defined by reduction of at least 2 points on an 8-category ordinal scale of clinical improvement or discharge from hospital, whichever comes first; Change from baseline in cardiac markers high sensitivity troponin I (hsTnI) and NT-proBNP; Change from baseline in TNF alpha, IL-1, IL-6, and other inflammatory biomarkers; and Incidence and severity of any treatment emergent adverse events (TEAEs) or serious adverse events (SAEs) as determined by clinical AEs, vital sign, laboratory, ECG abnormalities, and their relationship to PB1046 and immunogenicity of PB1046.

Exploratory Objectives: To assess the change in hemodynamic parameters, such as, pulmonary artery pressure and cardiac output, in COVID-19 patients requiring right-heart catheterization; To assess the impact of PB1046 on development of multi-system organ failure (MSOF), or need for ECMO; To assess the effect of PB1046 on ferritin, D-dimer, liver function, and other blood chemistry, hematology, and coagulation markers.

Exploratory Endpoints: Change in invasive hemodynamic parameters, including mean PA pressure and cardiac output, in patients requiring right-heart catheterization; Development of multi-system organ failure (MSOF) and number of MSOF-free days; Change in circulating ferritin, D-dimer, and other blood chemistry and coagulation markers tested locally; and Incidence of patients requiring ECMO.

Inclusion Criteria: Male or female 18-85 years old hospitalized COVID-19 patients (e.g. having a positive local SARS-CoV2 test) will be included in this trial. Patients receiving oxygen by face mask or nasal cannula/prongs and/or with elevated markers of cardiac injury or dysfunction (hsTnl or TN-proBNP) as assessed by local testing.

Exclusion Criteria: Subjects will be excluded from the study if they meet any of the following criteria: patients considered unsalavageable or expected to expire within 24 hours; on mechanical ventilation or imminent need for mechanical ventilation expected in the next 24 hours; evidence of acute end-organ injury in 2 or more organ systems (not including cardiac or pulmonary) such as renal, hepatic, or CNS injury; receiving another investigational therapy for treatment or prevention of COVID-19-related hypoxemic respiratory failure or ARDS other than antiviral therapy; systolic blood pressure (SBP)<95 mmHg and/or diastolic blood pressure (DBP)<50 mmHg or overt symptomatic hypotension during screening; resting heart rate>110 BPM (beats per minute) during screening; severe chronic renal failure as measured by the estimated glomerular filtration rate (eGFR) of<30 mL/min/1.73 m² using the local laboratory calculation of eGFR; significant liver dysfunction as measured by any one of the following at screening: ALT (alanine transaminase)>3.0 times ULN (upper limit of normal); AST (aspartate transaminase)>3.0 times ULN; serum bilirubin≥1.6 mg/dL; any in-patient surgical procedure or hospitalization (defined as>23 hours) within 30 days of subject screening except for prior hospitalization for COVID-19; known hypersensitivity to study drug or any of the excipients of the drug formulation; pregnant or lactating female subjects; any other condition which, in the opinion of the Investigator, would place the subject at increased risk or would preclude obtaining informed consent or confound the objectives of the study.

### INCORPORATION BY REFERENCE

All publications, patents, and patent publications cited are incorporated by reference herein in their entirety for all purposes.

This application incorporates by reference the following publications in their entireties for all purposes: U.S. 2001/0034050; U.S. 2009/0220455; U.S. Pat. No. 8,334,257; U.S. 2013/0310538 ; U.S. 2013/0172274; U.S. 2011/0236384; U.S. Pat. Nos. 6,582,926; 7,429,458; 7,364,859; 8,178,495; U.S. 2013/0079277; U.S. 2013/0085099; U.S. 2013/0143802; U.S. 2014/0024600; U.S. 2011/0178017; U.S. Pat. No. 7,709,227; U.S. 2011/0123487; U.S. Pat. No. 8,729,018; U.S. 2014/0171370; U.S. 2013/0150291; WO/2014/113434; U.S. 2014/0213516; WO 2016/130518, and U.S. Application No. 62/082,945 filed Nov. 21, 2014.

### REFERENCES

1. Wu C, Chen X, Cai Y, et al. Risk Factors Associated With Acute Respiratory Distress Syndrome and Death in Patients With Coronavirus Disease 2019 Pneumonia in Wuhan, China. JAMA Intern Med. 2020 Mar. 13

2. Zhang, Y. F et al. Vasoactive intestinal peptide inhibits the activation of murine fibroblasts and expression of interleukin 17 receptor C. Cell Biol Int 2019 43, 770-780

3. Szema A M, Hamidi S A. Gene deletion of VIP leads to increased mortality associated with progressive right ventricular hypertrophy. J Cardiovasc Dis. 2014 Apr. 1; 2(3):131-6.

4. Delgado M, Pozo D, Martinez C, et al. Vasoactive Intestinal Peptide and Pituitary Adenylate Cyclase-Activating Polypeptide Inhibit Endotoxin-Induced TNF-a Production by Macrophages: In Vitro and In Vivo Studies. The Journal of Immunology, 1999 162: 2358-2367

5. Berisha H, Foda H, Sakakibara, H et al. Vasoactive Intestinal Peptide Prevents Lung Injury due to xanthine/xanthine oxidase. Am J Physiol. 1990 August; 259(2 Pt 1):L151-5.

6. Sakakibara H, Shima K, Said S I. 1994. Characterization of vasoactive intestinal peptide receptors on rat alveolar macrophages. Am J Physiol. 1994 September; 267(3 Pt 1):L256-62.

7. Ran W Z, Dong L, Tang C Y Zhou Y, et al Vasoactive intestinal peptide suppresses macrophage-mediated inflammation by downregulating interleukin-17A expression via PKA- and PKC-dependent pathways. Int J Exp Pathol. 2015 August; 96(4):269-75.

8. Sun G Y, Yang H H, Guan X X et al. Vasoactive intestinal peptide overexpression mediated by lentivirus attenuates lipopolysaccharide-induced acute lung injury in mice by inhibiting inflammation. Mol Immunol. 2018 May; 97:8-15.

9. Zhou Y, Zhang C Y, Duan J X. Vasoactive intestinal peptide suppresses the NLRP3 inflammasome activation in lipopolysaccharide-induced acute lung injury mice and macrophages. Biomed Pharmacother, 2020 January; 121:109596

10. Petkov V, Mosgoeller W, et al. Vasoactive intestinal peptide as a new drug for treatment of primary pulmonary hypertension. Journal of Clinical Investigation 2003; 111: 1339-1346.

11. Burian B, Storka A, Nadler B et al. Inhaled Vasoactive Intestinal Peptide Improves 6 minute Walk Test and Quality of Life in Patients with COPD: The VIP/COPD Trial, Chest. 2006 Volume 130.

12. Mehta P, McAuley D, Brown M, et al. COVID-19: Consider cytokine storm syndromes and immunosuppression. The Lancet. Vol 395, Issue 10229, P1033-1034, Mar. 25, 2020

13. Richardson S, Hirsch J S, Narasimhan M et al. Presenting Characteristics, Comorbidities, and Outcomes Among 5700 Patients Hospitalized With COVID-19 in the New York City Area. JAMA Published online Apr. 22, 2020. doi:10.1001/jama.2020.6775

1. A method of treating inflammatory lung disease comprising administering a pharmaceutical composition comprising Vasoactive Intestinal Peptide (VIP) and an elastin-like peptide (ELP) to a patient in need thereof.

2. The method of claim 1, wherein the pharmaceutical composition comprises the polypeptide of SEQ ID NO: 3.

3. The method of claim 1 or 2, wherein the patient is infected by, or presumed to be infected by, a coronavirus.

4. The method of any of claims 1-3, wherein the patient is infected by, or presumed to be infected by, SARS-CoV-2, SARS, or MERS.

5. The method of claim 4, wherein the patient has developed COVID-19 or symptoms thereof.

6. The method of claim 5, wherein the patient has developed severe COVID-19 or symptoms thereof.

7. The method of claim 5, wherein the patient is at high risk of developing severe COVID-19 or symptoms thereof.

8. The method of any one of claims 1-7, wherein the patient presents with a comorbidity.

9. The method of claim 8, wherein the comorbidity increases the risk of the patient developing severe COVID-19 or symptoms thereof.

10. The method of claim 9, wherein the comorbidity is selected from the group consisting of: obesity, hypertension, diabetes, an autoimmune disorder (e.g. rheumatoid arthritis), heart disease, heart failure, atherosclerosis, cancer (e.g. lung cancer), a history of smoking or exposure to other lung-damaging agents), liver disease, alcoholism, other pulmonary infection, and chronic kidney disease.

11. The method of any one of claims 1-9, wherein one or more factors increasing patient risk of developing severe COVID-19 is race and/or socioeconomic status.

12. The method of any one of claims 1-11, wherein the patient presents with elevated markers of cardiac injury or dysfunction.

13. The method of any one of claims 1-12, wherein the patient presents with one or more of the following symptoms:
a) low oxygen saturation levels;
b) increased respiration rate;
c) requires oxygen therapy;
d) requires a ventilator to breathe; and
e) fever.

14. The method of any one of claims 1-13, wherein the patient is administered a low dose of the pharmaceutical composition comprising the polypeptide of SEQ ID NO: 3.

15. The method of claim 14, wherein the patient is administered a dose of about 10 mg of the pharmaceutical composition comprising the polypeptide of SEQ ID NO: 3.

16. The method of claim 15, wherein the patient is administered a low dose of the pharmaceutical composition comprising the polypeptide of SEQ ID NO: 3 for four weeks or until hospital discharge.

17. The method of any one of claims 1-13, wherein the patient is administered a moderate dose of the pharmaceutical composition comprising the polypeptide of SEQ ID NO: 3.

18. The method of claim 17, wherein the patient is administered a dose of about 40 mg of the pharmaceutical composition comprising the polypeptide of SEQ ID NO: 3.

19. The method of claim 18, wherein the patient is administered a moderate dose of the pharmaceutical composition comprising the polypeptide of SEQ ID NO: 3 for four weeks or until hospital discharge.

20. The method of any one of claims 1-13, wherein the patient is administered a high dose of the pharmaceutical composition comprising the polypeptide of SEQ ID NO: 3.

21. The method of claim 20, wherein the patient is administered a dose of about 100 mg of the pharmaceutical composition comprising the polypeptide of SEQ ID NO: 3.

22. The method of claim 21, wherein the patient is administered a high dose of the pharmaceutical composition comprising the polypeptide of SEQ ID NO: 3 for four weeks or until hospital discharge.

23. The method of any one of claims 1-22, wherein the pharmaceutical composition comprising the polypeptide of SEQ ID NO: 3 is administered subcutaneously.

24. A method of treating a patient exhibiting one or more symptoms of SARS-CoV-2 infection, comprising administering and effective amount of a pharmaceutical composition comprising a Vasoactive Intestinal Peptide and an Elastin-like peptide (ELP).

25. The method of claim 24, wherein the pharmaceutical composition is administered prior to the development of Acute Respiratory Distress Syndrome (ARDS) in the patient.

26. The method of claim 25, wherein the pharmaceutical composition is administered when the patient is exhibiting one or more symptoms of ARDS.

27. The method of any one of claims 24 to 26, wherein administration of the pharmaceutical composition prevents the onset or progression of ARDS in the patient.

28. The method of any one of claims 24 to 27, wherein the pharmaceutical composition comprises the polypeptide of SEQ ID NO: 3.

29. The method of any one of claims 24-28, wherein the patient presents with a comorbidity.

30. The method of claim 29, wherein the comorbidity increases the risk of the patient developing ARDS or symptoms thereof.

31. The method of claim 30, wherein the comorbidity is selected from the group consisting of: obesity, hypertension, diabetes, an autoimmune disorder (e.g. rheumatoid arthritis), heart disease, heart failure, atherosclerosis, cancer (e.g. lung cancer), a history of smoking or exposure to other lung-damaging agents), liver disease, alcoholism, other pulmonary infection, and chronic kidney disease.

32. The method of any one of claims 29-30, wherein one or more factors increasing patient risk of developing ARDS is race and/or socioeconomic status.

33. The method of any one of claims 24 to 32, wherein the patient presents with elevated markers of cardiac injury or dysfunction.

34. The method of any one of claims 24-33, wherein the patient presents with one or more of the following symptoms:
a) low oxygen saturation levels;
b) increased respiration rate;
c) requires oxygen therapy;
d) requires a ventilator to breathe; and
e) fever.

35. The method of any one of claims 24-34, wherein the patient is administered a low dose of the pharmaceutical composition comprising the polypeptide of SEQ ID NO: 3.

36. The method of claim 35, wherein the patient is administered a dose of about 10 mg of the pharmaceutical composition comprising the polypeptide of SEQ ID NO: 3.

37. The method of claim 36, wherein the patient is administered a low dose of the pharmaceutical composition comprising the polypeptide of SEQ ID NO: 3 for four weeks or until hospital discharge.

38. The method of any one of claims 24-34, wherein the patient is administered a moderate dose of the pharmaceutical composition comprising the polypeptide of SEQ ID NO: 3.

39. The method of claim 38, wherein the patient is administered a dose of about 40 mg of the pharmaceutical composition comprising the polypeptide of SEQ ID NO: 3.

40. The method of claim 39, wherein the patient is administered a moderate dose of the pharmaceutical composition comprising the polypeptide of SEQ ID NO: 3 for four weeks or until hospital discharge.

41. The method of any one of claims 24-34, wherein the patient is administered a high dose of the pharmaceutical composition comprising the polypeptide of SEQ ID NO: 3.

42. The method of claim 41, wherein the patient is administered a dose of about 100 mg of the pharmaceutical composition comprising the polypeptide of SEQ ID NO: 3.

43. The method of claim 42, wherein the patient is administered a high dose of the pharmaceutical composition comprising the polypeptide of SEQ ID NO: 3 for four weeks or until hospital discharge.

44. The method of any one of claims 24-43, wherein the pharmaceutical composition comprising the polypeptide of SEQ ID NO: 3 is administered subcutaneously.


SEQUENCE LISTING (8 sequences)

SEQ ID NO: 1 - protein, 5 aa, Artificial Sequence
  ELP component sequence
  VARIANT (4)..(4): Xaa can be any naturally occurring or non- natural amino acid
VPGXG

SEQ ID NO: 2 - protein, 29 aa, Artificial Sequence
  M-VIP
MHSDAVFTDNYTRLRKQMAVKKYLNSILN

SEQ ID NO: 3 - protein, 634 aa, Artificial Sequence
  M-VIP ELP1-120
MHSDAVFTDNYTRLRKQMAVKKYLNSILNVPGVGVPGVGVPGGGVPGAGVPGVGVPGVGVPGVGVPGGGVPGAGVPGGGVPGVGVPGVGVPGGGVPGAGVPGVGVPGVGVPGVGVPGGGVPGAGVPGGGVPGVGVPGVGVPGGGVPGAGVPGVGVPGVGVPGVGVPGGGVPGAGVPGGGVPGVGVPGVGVPGGGVPGAGVPGVGVPGVGVPGVGVPGGGVPGAGVPGGGVPGVGVPGVGVPGGGVPGAGVPGVGVPGVGVPGVGVPGGGVPGAGVPGGGVPGVGVPGVGVPGGGVPGAGVPGVGVPGVGVPGVGVPGGGVPGAGVPGGGVPGVGVPGVGVPGGGVPGAGVPGVGVPGVGVPGVGVPGGGVPGAGVPGGGVPGVGVPGVGVPGGGVPGAGVPGVGVPGVGVPGVGVPGGGVPGAGVPGGGVPGVGVPGVGVPGGGVPGAGVPGVGVPGVGVPGVGVPGGGVPGAGVPGGGVPGVGVPGVGVPGGGVPGAGVPGVGVPGVGVPGVGVPGGGVPGAGVPGGGVPGVGVPGVGVPGGGVPGAGVPGVGVPGVGVPGVGVPGGGVPGAGVPGGGVPGVGVPGVGVPGGGVPGAGVPGVGVPGVGVPGVGVPGGGVPGAGVPGGGVPGWP

SEQ ID NO: 4 - protein, 636 aa, Artificial Sequence
  MAA-VIP ELP1-120
MAAHSDAVFTDNYTRLRKQMAVKKYLNSILNVPGVGVPGVGVPGGGVPGAGVPGVGVPGVGVPGVGVPGGGVPGAGVPGGGVPGVGVPGVGVPGGGVPGAGVPGVGVPGVGVPGVGVPGGGVPGAGVPGGGVPGVGVPGVGVPGGGVPGAGVPGVGVPGVGVPGVGVPGGGVPGAGVPGGGVPGVGVPGVGVPGGGVPGAGVPGVGVPGVGVPGVGVPGGGVPGAGVPGGGVPGVGVPGVGVPGGGVPGAGVPGVGVPGVGVPGVGVPGGGVPGAGVPGGGVPGVGVPGVGVPGGGVPGAGVPGVGVPGVGVPGVGVPGGGVPGAGVPGGGVPGVGVPGVGVPGGGVPGAGVPGVGVPGVGVPGVGVPGGGVPGAGVPGGGVPGVGVPGVGVPGGGVPGAGVPGVGVPGVGVPGVGVPGGGVPGAGVPGGGVPGVGVPGVGVPGGGVPGAGVPGVGVPGVGVPGVGVPGGGVPGAGVPGGGVPGVGVPGVGVPGGGVPGAGVPGVGVPGVGVPGVGVPGGGVPGAGVPGGGVPGVGVPGVGVPGGGVPGAGVPGVGVPGVGVPGVGVPGGGVPGAGVPGGGVPGVGVPGVGVPGGGVPGAGVPGVGVPGVGVPGVGVPGGGVPGAGVPGGGVPGWP

SEQ ID NO: 5 - protein, 28 aa, Homo sapiens
HSDAVFTDNYTRLRKQMAVKKYLNSILN

SEQ ID NO: 6 - protein, 31 aa, Artificial Sequence
  MAA-VIP
MAAHSDAVFTDNYTRLRKQMAVKKYLNSILN

SEQ ID NO: 7 - protein, 633 aa, Artificial Sequence
  VIP ELP1-120
HSDAVFTDNYTRLRKQMAVKKYLNSILNVPGVGVPGVGVPGGGVPGAGVPGVGVPGVGVPGVGVPGGGVPGAGVPGGGVPGVGVPGVGVPGGGVPGAGVPGVGVPGVGVPGVGVPGGGVPGAGVPGGGVPGVGVPGVGVPGGGVPGAGVPGVGVPGVGVPGVGVPGGGVPGAGVPGGGVPGVGVPGVGVPGGGVPGAGVPGVGVPGVGVPGVGVPGGGVPGAGVPGGGVPGVGVPGVGVPGGGVPGAGVPGVGVPGVGVPGVGVPGGGVPGAGVPGGGVPGVGVPGVGVPGGGVPGAGVPGVGVPGVGVPGVGVPGGGVPGAGVPGGGVPGVGVPGVGVPGGGVPGAGVPGVGVPGVGVPGVGVPGGGVPGAGVPGGGVPGVGVPGVGVPGGGVPGAGVPGVGVPGVGVPGVGVPGGGVPGAGVPGGGVPGVGVPGVGVPGGGVPGAGVPGVGVPGVGVPGVGVPGGGVPGAGVPGGGVPGVGVPGVGVPGGGVPGAGVPGVGVPGVGVPGVGVPGGGVPGAGVPGGGVPGVGVPGVGVPGGGVPGAGVPGVGVPGVGVPGVGVPGGGVPGAGVPGGGVPGVGVPGVGVPGGGVPGAGVPGVGVPGVGVPGVGVPGGGVPGAGVPGGGVPGWP

SEQ ID NO: 8 - protein, 605 aa, Artificial Sequence
  ELP1-120
VPGVGVPGVGVPGGGVPGAGVPGVGVPGVGVPGVGVPGGGVPGAGVPGGGVPGVGVPGVGVPGGGVPGAGVPGVGVPGVGVPGVGVPGGGVPGAGVPGGGVPGVGVPGVGVPGGGVPGAGVPGVGVPGVGVPGVGVPGGGVPGAGVPGGGVPGVGVPGVGVPGGGVPGAGVPGVGVPGVGVPGVGVPGGGVPGAGVPGGGVPGVGVPGVGVPGGGVPGAGVPGVGVPGVGVPGVGVPGGGVPGAGVPGGGVPGVGVPGVGVPGGGVPGAGVPGVGVPGVGVPGVGVPGGGVPGAGVPGGGVPGVGVPGVGVPGGGVPGAGVPGVGVPGVGVPGVGVPGGGVPGAGVPGGGVPGVGVPGVGVPGGGVPGAGVPGVGVPGVGVPGVGVPGGGVPGAGVPGGGVPGVGVPGVGVPGGGVPGAGVPGVGVPGVGVPGVGVPGGGVPGAGVPGGGVPGVGVPGVGVPGGGVPGAGVPGVGVPGVGVPGVGVPGGGVPGAGVPGGGVPGVGVPGVGVPGGGVPGAGVPGVGVPGVGVPGVGVPGGGVPGAGVPGGGVPGVGVPGVGVPGGGVPGAGVPGVGVPGVGVPGVGVPGGGVPGAGVPGGGVPGWP